SEQUENCE LISTING (8 sequences)

SEQ ID NO: 1 - protein, 9 aa, Artificial
  epitope recognized by a Tcell receptor
HEYPVGSQL

SEQ ID NO: 2 - protein, 15 aa, Artificial
  epitope recognized by a Tcell receptor
AELIEANLLWRQEMG

SEQ ID NO: 3 - DNA, 546 bp, Artificial
  DNA sequence of a representative native, unmodified NS3 protease domain
atggcgcccatcacggcgtacgcccagcagacaaggggcctcctagggtgcataatcaccagcctaactggccgggacaaaaaccaagtggagggtgaggtccagattgtgtcaactgctgcccaaaccttcctggcaacgtgcatcaatggggtgtgctggactgtctaccacggggccggaacgaggaccatcgcgtcacccaagggtcctgtcatccagatgtataccaatgtagaccaagaccttgtgggctggcccgctccgcaaggtagccgatcattgacaccctgcacttgcggctcctcggacctttacctggtcacgaggcacgccgatgtcattcccgtgcgccggcggggtgatagcaggggcagcctgctgtcgccccggcccatttcctacttgaaaggctcctcggggggtccgctgttgtgccccgcggggcacgccgtgggcatatttagggccgcggtgtgcacccgtggagtggctaaggcggtggactttatccctgtggagaacctagagacaaccatgaggtcc

SEQ ID NO: 4 - protein, 182 aa, Artificial
  amino acid sequence of a representative native unmodified NS3 protease domain
MAPITAYAQQTRGLLGCIITSLTGRDKNQVEGEVQIVSTAAQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGIFRAAVCTRGVAKAVDFIPVENLETTMRS

SEQ ID NO: 5 - DNA, 5676 bp, Artificial
  DNA sequence of a representative modified fusion protein, with the NS3 protease domain deleted from the N-terminus and including amino acids 1-121 of Core on the C-terminus
atggctgcatatgcagctcagggctataaggtgctagtactcaacccctctgttgctgcaacactgggctttggtgcttacatgtccaaggctcatgggatcgatcctaacatcaggaccggggtgagaacaattaccactggcagccccatcacgtactccacctacggcaagttccttgccgacggcgggtgctcggggggcgcttatgacataataatttgtgacgagtgccactccacggatgccacatccatcttgggcattggcactgtccttgaccaagcagagactgcgggggcgagactggttgtgctcgccaccgccacccctccgggctccgtcactgtgccccatcccaacatcgaggaggttgctctgtccaccaccggagagatccctttttacggcaaggctatccccctcgaagtaatcaagggggggagacatctcatcttctgtcattcaaagaagaagtgcgacgaactcgccgcaaagctggtcgcattgggcatcaatgccgtggcctactaccgcggtcttgacgtgtccgtcatcccgaccagcggcgatgttgtcgtcgtggcaaccgatgccctcatgaccggctataccggcgacttcgactcggtgatagactgcaatacgtgtgtcacccagacagtcgatttcagccttgaccctaccttcaccattgagacaatcacgctcccccaagatgctgtctcccgcactcaacgtcggggcaggactggcagggggaagccaggcatctacagatttgtggcaccgggggagcgcccctccggcatgttcgactcgtccgtcctctgtgagtgctatgacgcaggctgtgcttggtatgagctcacgcccgccgagactacagttaggctacgagcgtacatgaacaccccggggcttcccgtgtgccaggaccatcttgaattttgggagggcgtctttacaggcctcactcatatagatgcccactttctatcccagacaaagcagagtggggagaaccttccttacctggtagcgtaccaagccaccgtgtgcgctagggctcaagcccctcccccatcgtgggaccagatgtggaagtgtttgattcgcctcaagcccaccctccatgggccaacacccctgctatacagactgggcgctgttcagaatgaaatcaccctgacgcacccagtcaccaaatacatcatgacatgcatgtcggccgacctggaggtcgtcacgagcacctgggtgctcgttggcggcgtcctggctgctttggccgcgtattgcctgtcaacaggctgcgtggtcatagtgggcagggtcgtcttgtccgggaagccggcaatcatacctgacagggaagtcctctaccgagagttcgatgagatggaagagtgctctcagcacttaccgtacatcgagcaagggatgatgctcgccgagcagttcaagcagaaggccctcggcctcctgcagaccgcgtcccgtcaggcagaggttatcgcccctgctgtccagaccaactggcaaaaactcgagaccttctgggcgaagcatatgtggaacttcatcagtgggatacaatacttggcgggcttgtcaacgctgcctggtaaccccgccattgcttcattgatggcttttacagctgctgtcaccagcccactaaccactagccaaaccctcctcttcaacatattgggggggtgggtggctgcccagctcgccgcccccggtgccgctactgcctttgtgggcgctggcttagctggcgccgccatcggcagtgttggactggggaaggtcctcatagacatccttgcagggtatggcgcgggcgtggcgggagctcttgtggcattcaagatcatgagcggtgaggtcccctccacggaggacctggtcaatctactgcccgccatcctctcgcccggagccctcgtagtcggcgtggtctgtgcagcaatactgcgccggcacgttggcccgggcgagggggcagtgcagtggatgaaccggctgatagccttcgcctcccgggggaaccatgtttcccccacgcactacgtgccggagagcgatgcagctgcccgcgtcactgccatactcagcagcctcactgtaacccagctcctgaggcgactgcaccagtggataagctcggagtgtaccactccatgctccggttcctggctaagggacatctgggactggatatgcgaggtgttgagcgactttaagacctggctaaaagctaagctcatgccacagctgcctgggatcccctttgtgtcctgccagcgcgggtataagggggtctggcgaggggacggcatcatgcacactcgctgccactgtggagctgagatcactggacatgtcaaaaacgggacgatgaggatcgtcggtcctaggacctgcaggaacatgtggagtgggaccttccccattaatgcctacaccacgggcccctgtaccccccttcctgcgccgaactacacgttcgcgctatggagggtgtctgcagaggaatacgtggagataaggcaggtgggggacttccactacgtgacgggtatgactactgacaatcttaaatgcccgtgccaggtcccatcgcccgaatttttcacagaattggacggggtgcgcctacataggtttgcgcccccctgcaagcccttgctgcgggaggaggtatcattcagagtaggactccacgaatacccggtagggtcgcaattaccttgcgagcccgaaccggacgtggccgtgttgacgtccatgctcactgatccctcccatataacagcagaggcggccgggcgaaggttggcgaggggatcacccccctctgtggccagctcctcggctagccagctatccgctccatctctcaaggcaacttgcaccgctaaccatgactcccctgatgctgagctcatagaggccaacctcctatggaggcaggagatgggcggcaacatcaccagggttgagtcagaaaacaaagtggtgattctggactccttcgatccgcttgtggcggaggaggacgagcgggagatctccgtacccgcagaaatcctgcggaagtctcggagattcgcccaggccctgcccgtttgggcgcggccggactataaccccccgctagtggagacgtggaaaaagcccgactacgaaccacctgtggtccatggctgcccgcttccacctccaaagtcccctcctgtgcctccgcctcggaagaagcggacggtggtcctcactgaatcaaccctatctactgccttggccgagctcgccaccagaagctttggcagctcctcaacttccggcattacgggcgacaatacgacaacatcctctgagcccgccccttctggctgcccccccgactccgacgctgagtcctattcctccatgccccccctggagggggagcctggggatccggatcttagcgacgggtcatggtcaacggtcagtagtgaggccaacgcggaggatgtcgtgtgctgctcaatgtcttactcttggacaggcgcactcgtcaccccgtgcgccgcggaagaacagaaactgcccatcaatgcactaagcaactcgttgctacgtcaccacaatttggtgtattccaccacctcacgcagtgcttgccaaaggcagaagaaagtcacatttgacagactgcaagttctggacagccattaccaggacgtactcaaggaggttaaagcagcggcgtcaaaagtgaaggctaacttgctatccgtagaggaagcttgcagcctgacgcccccacactcagccaaatccaagtttggttatggggcaaaagacgtccgttgccatgccagaaaggccgtaacccacatcaactccgtgtggaaagaccttctggaagacaatgtaacaccaatagacactaccatcatggctaagaacgaggttttctgcgttcagcctgagaaggggggtcgtaagccagctcgtctcatcgtgttccccgatctgggcgtgcgcgtgtgcgaaaagatggctttgtacgacgtggttacaaagctccccttggccgtgatgggaagctcctacggattccaatactcaccaggacagcgggttgaattcctcgtgcaagcgtggaagtccaagaaaaccccaatggggttctcgtatgatacccgctgctttgactccacagtcactgagagcgacatccgtacggaggaggcaatctaccaatgttgtgacctcgacccccaagcccgcgtggccatcaagtccctcaccgagaggctttatgttgggggccctcttaccaattcaaggggggagaactgcggctatcgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagcgcgggggtccaggaggacgcggcgagcctgagagccttcacggaggctatgaccaggtactccgccccccctggggaccccccacaaccagaatacgacttggagctcataacatcatgctcctccaacgtgtcagtcgcccacgacggcgctggaaagagggtctactacctcacccgtgaccctacaacccccctcgcgagagctgcgtgggagacagcaagacacactccagtcaattcctggctaggcaacataatcatgtttgcccccacactgtgggcgaggatgatactgatgacccatttctttagcgtccttatagccagggaccagcttgaacaggccctcgattgcgagatctacggggcctgctactccatagaaccactggatctacctccaatcattcaaagactccatggcctcagcgcattttcactccacagttactctccaggtgaaatcaatagggtggccgcatgcctcagaaaacttggggtaccgcccttgcgagcttggagacaccgggcccggagcgtccgcgctaggcttctggccagaggaggcagggctgccatatgtggcaagtacctcttcaactgggcagtaagaacaaagctcaaactcactccaatagcggccgctggccagctggacttgtccggctggttcacggctggctacagcgggggagacatttatcacagcgtgtctcatgcccggccccgctggatctggttttgcctactcctgcttgctgcaggggtaggcatctacctcctccccaaccgaatgagcacgaatcctaaacctcaaagaaagaccaaacgtaacaccaaccggcggccgcaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaag

SEQ ID NO: 6 - protein, 1892 aa, Artificial
  amino acid sequence of a representative modified fusion protein, with the NS3 protease domain deleted from the N-terminus and including amino acids 1-121 of Core on the C-terminus
MAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEITLTHPVTKYIMTCMSADLEVVTSTWVLVGGVLAALAAYCLSTGCVVIVGRVVLSGKPAIIPDREVLYREFDEMEECSQHLPYIEQGMMLAEQFKQKALGLLQTASRQAEVIAPAVQTNWQKLETFWAKHMWNFISGIQYLAGLSTLPGNPAIASLMAFTAAVTSPLTTSQTLLFNILGGWVAAQLAAPGAATAFVGAGLAGAAIGSVGLGKVLIDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYKGVWRGDGIMHTRCHCGAEITGHVKNGTMRIVGPRTCRNMWSGTFPINAYTTGPCTPLPAPNYTFALWRVSAEEYVEIRQVGDFHYVTGMTTDNLKCPCQVPSPEFFTELDGVRLHRFAPPCKPLLREEVSFRVGLHEYPVGSQLPCEPEPDVAVLTSMLTDPSHITAEAAGRRLARGSPPSVASSSASQLSAPSLKATCTANHDSPDAELIEANLLWRQEMGGNITRVESENKVVILDSFDPLVAEEDEREISVPAEILRKSRRFAQALPVWARPDYNPPLVETWKKPDYEPPVVHGCPLPPPKSPPVPPPRKKRTVVLTESTLSTALAELATRSFGSSSTSGITGDNTTTSSEPAPSGCPPDSDAESYSSMPPLEGEPGDPDLSDGSWSTVSSEANAEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVTHINSVWKDLLEDNVTPIDTTIMAKNEVFCVQPEKGGRKPARLIVFPDLGVRVCEKMALYDVVTKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLARGGRAAICGKYLFNWAVRTKLKLTPIAAAGQLDLSGWFTAGYSGGDIYHSVSHARPRWIWFCLLLLAAGVGIYLLPNRMSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGK

SEQ ID NO: 7 - protein, 21 aa, Artificial
  E2 epitope consensus sequence
GSAARTTSGFVSLFAPGAKQN

SEQ ID NO: 8 - protein, 23 aa, Artificial
  NS4A peptide
KKGSVVIVGRIVLSGKPAIIPKK

(19) United States
(12) Patent Application Publication
(10) Pub. No.: US 2005/0074465 A1
(43) Pub. Date: Apr. 7, 2005

(54) HCV fusion proteins with modified NS3 domains

(76) Inventor: Michael Houghton, Danville, CA (US)

Correspondence Address: Chiron Corporation;Intellectual Property - R440, P.O. Box 8097, Emeryville, CA, 94662-8097, US

(21) Appl. No.: 10/612,884

(22) Filed: Jul. 2, 2003

Related U.S. Application Data
(63) Continuation-in-part of application No. 09/721,479, filed on Nov. 22, 2000.
(60) Provisional application No. 60/167,502, filed on Nov. 24, 1999.
(60) Provisional application No. 60/393,694, filed on Jul. 2, 2002.
(60) Provisional application No. 60/394,510, filed on Jul. 8, 2002.

(51) Int. Cl. A61K 39/29; C07H 21/04; C12P 21/04; C07K 14/02

(52) U.S. Cl. 424/189.1; 435/69.7; 435/320.1; 435/325; 530/350; 536/23.72

(57) ABSTRACT

The invention provides HCV fusion proteins that include a mutated NS3 protease domain, fused to at least one other HCV epitope derived from another region of the HCV polyprotein. The fusions can be used in methods of stimulating a cellular immune response to HCV, such as activating hepatitis C virus (HCV)-specific T cells, including CD4⁺ and CD8⁺ T cells. The method can be used in model systems to develop HCV-specific immunogenic compositions, as well as to immunize a mammal against HCV.

### CROSS-REFERENCE TO RELATED APPLICATION

This application is a continuation-in-part of application Ser. No. 09/721,479, filed Nov. 22, 2000, from which application priority is claimed under 35 USC §120, and which is related to provisional application No. 60/167,502, filed Nov. 24, 1999, from which application priority is claimed under 35 USC §119(e)(1) and which applications are incorporated herein by reference in their entireties. This application is also related to application Ser. Nos. 60/393,694, filed Jul. 2, 2002, and 60/394,510, filed Jul. 8, 2002, from which applications priority is claimed under 35 USC §119(e)(1) and which applications are incorporated herein by reference in their entireties.

### TECHNICAL FIELD

The present invention relates to hepatitis C virus (HCV) constructs. More particularly, the invention relates to HCV fusion proteins with modified NS3 domains. The proteins are capable of stimulating cell-mediated immune responses, such as for priming and/or activating HCV-specific T cells.

### BACKGROUND OF THE INVENTION

Hepatitis C virus (HCV) infection is an important health problem with approximately 1% of the world's population infected with the virus. Over 75% of acutely infected individuals eventually progress to a chronic carrier state that can result in cirrhosis, liver failure, and hepatocellular carcinoma. See, Alter et al. (1992) N. Engl. J. Med. 327:1899-1905; Resnick and Koff. (1993) Arch. Intem. Med. 153:1672-1677; Seeff (1995) Gastrointest. Dis. 6:20-27; Tong et al. (1995) N. Engl. J. Med. 332:1463-1466.

HCV was first identified and characterized as a cause of NANBH by Houghton et al. The viral genomic sequence of HCV is known, as are methods for obtaining the sequence. See, e.g., International Publication Nos. WO 89/04669; WO 90/11089; and WO 90/14436. HCV has a 9.5 kb positive-sense, single-stranded RNA genome and is a member of the Flaviridae family of viruses. At least six distinct, but related genotypes of HCV, based on phylogenetic analyses, have been identified (Simmonds et al., J. Gen. Virol. (1993) 74:2391-2399). The virus encodes a single polyprotein having more than 3000 amino acid residues (Choo et al., Science (1989) 244:359-362; Choo et al., Proc. Natl. Acad. Sci. USA (1991) 88:2451-2455; Han et al., Proc. Natl. Acad. Sci. USA (1991) 88:1711-1715). The polyprotein is processed co- and post-translationally into both structural and non-structural (NS) proteins.

In particular, as shown in FIG. 1, several proteins are encoded by the HCV genome. The order and nomenclature of the cleavage products of the HCV polyprotein is as follows: NH₂₋C-E1-E2-p7-NS2-NS3-NS4a-NS4b-NS5a-NS5b-COOH. Initial cleavage of the polyprotein is catalyzed by host proteases which liberate three structural proteins, the N-terminal nucleocapsid protein (termed “core”) and two envelope glycoproteins, “E1” (also known as E) and “E2” (also known as E2/NS1), as well as nonstructural (NS) proteins that contain the viral enzymes. The NS regions are termed NS2, NS3, NS4 and NS5. NS2 is an integral membrane protein with proteolytic activity and, in combination with NS3, cleaves the NS2-NS3 sissle bond which in turn generates the NS3 N-terminus and releases a large polyprotein that includes both serine protease and RNA helicase activities. The NS3 protease serves to process the remaining polyprotein. In these reactions, NS3 liberates an NS3 cofactor (NS4a), two proteins (NS4b and NS5a), and an RNA-dependent RNA polymerase (NS5b). Completion of polyprotein maturation is initiated by autocatalytic cleavage at the NS3-NS4a junction, catalyzed by the NS3 serine protease.

Despite extensive advances in the development of pharmaceuticals against certain viruses like HIV, control of acute and chronic HCV infection has had limited success (Hoofnagle and di Bisceglie (1997) N. Engl. J. Med. 336:347-356). In particular, generation of cellular immune responses, such as strong cytotoxic T lymphocyte (CTL) responses, is thought to be important for the control and eradication of HCV infections. Thus, there is a need in the art for effective methods of stimulating cellular immune responses to HCV.

### SUMMARY OF THE INVENTION

It is an object of the invention to provide reagents and methods for stimulating a cellular immune response to HCV, such as priming and/or activating T cells which recognize epitopes of HCV polypeptides. This and other objects of the invention are provided by one or more of the embodiments described below.

The invention provides HCV fusion proteins useful for stimulating such responses. One embodiment of the invention is directed to an HCV fusion protein that includes an NS3 polypeptide modified to inhibit protease activity, such that cleavage of the fusion is inhibited. The fusion protein includes, in addition to the modified NS3 polypeptide, one or more polypeptides from other regions of an HCV polyprotein, described in further detail below. These polypeptides are derived from the same HCV isolate as the NS3 polypeptide, or from different strains and isolates including isolates having any of the various HCV genotypes, to provide increased protection against a broad range of HCV genotypes.

In certain embodiments, the modification to NS3 comprises a substitution of an amino acid corresponding to His-1083, Asp-1105 and/or Ser-1165, numbered relative to the full-length HCV-1 polyprotein.

In further embodiments, the protein comprises a modified NS3 polypeptide, an NS4 polypeptide, an NS5a polypeptide, and optionally a core polypeptide.

In additional embodiments, the protein further comprises an NS5b polypeptide, and optionally a core polypeptide.

In yet additional embodiments, the protein further comprises an E2 polypeptide, a p7 polypeptide, an NS2 polypeptide, and optionally a core polypeptide.

In further embodiments, the protein further comprises an E1 polypeptide, an E2 polypeptide, a p7 polypeptide, an NS2 polypeptide, and optionally a core polypeptide.

In additional embodiments, the protein further comprises an E2 polypeptide, and optionally a core polypeptide.

In yet further embodiments, the protein further comprises an E1 polypeptide, an E2 polypeptide, and optionally a core polypeptide.

In further embodiments, the protein comprises an E2 polypeptide, a modified NS3 polypeptide, and optionally a core polypeptide.

In additional embodiments, the protein comprises an E1 polypeptide, an E2 polypeptide, a modified NS3 polypeptide, and optionally a core polypeptide.

Another embodiment provides a fusion protein that consists essentially of a modified NS3, an NS4, an NS5a, and, optionally, a core polypeptide of an HCV. In certain embodiments, an NS5b polypeptide is also present.

In the embodiments above, the various regions in the fusion protein need not be in the order in which they naturally occur in the native HCV polyprotein. Thus, for example, the core polypeptide, if present, may be at the N- and/or C-terminus of the fusion.

In yet additional embodiments, the invention is directed to an immunogenic fusion protein consisting essentially of, in amino terminal to carboxy terminal direction:
(a) a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, and an NS5a polypeptide;
(b) a modified NS3 polypeptide comprising a substitution of an amino acid corresponding to His-1083, Asp- 1105 and/or Ser-1165, numbered relative to the full-length HCV-1 polyprotein such that protease activity is inhibited, an NS4 polypeptide, an NS5a polypeptide and an NS5b polypeptide;
(c) an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, and an NS5a polypeptide;
(d) an E1 polypeptide, an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, and an NS5a polypeptide;
(e) an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide and an NS5b polypeptide;
(f) an E1 polypeptide, an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide and an NS5b polypeptide;
(g) an E2 polypeptide and a modified NS3 polypeptide comprising substitution of an amino acid corresponding to His-1083, Asp-1105 and/or Ser-1165, numbered relative to the full-length HCV-1 polyprotein such that protease activity is inhibited;
(h) an E1 polypeptide, an E2 polypeptide and a modified NS3 polypeptide comprising a substitution of an amino acid corresponding to His-1083, Asp-1105 and/or Ser-1165, numbered relative to the full-length HCV-1 polyprotein such that protease activity is inhibited;
(i) an E2 polypeptide, a p7 polypeptide, an NS2 polypeptide and a modified NS3 polypeptide comprising a substitution of an amino acid corresponding to His-1083, Asp-1105 and/or Ser-1165, numbered relative to the full-length HCV-1 polyprotein such that protease activity is inhibited; or
(j) an E1 polypeptide, an E2 polypeptide, a p7 polypeptide, an NS2 polypeptide and a modified NS3 polypeptide comprising a substitution of an amino acid corresponding to His-1083, Asp-1105 and/or Ser-1165, numbered relative to the full-length HCV-1 polyprotein such that protease activity is inhibited.

In another embodiment, the invention is directed to an immunogenic fusion protein consisting essentially of, in amino terminal to carboxy terminal direction:
(a) a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide, and a core polypeptide;
(b) a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide, an NS5b polypeptide and a core polypeptide;
(c) an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide and a core polypeptide;
(d) an E1 polypeptide, an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide and a core polypeptide;
(e) an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide, an NS5b polypeptide and a core polypeptide;
(f) an E1 polypeptide, an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide, an NS5b polypeptide and a core polypeptide;
(g) an E2 polypeptide, a modified NS3 polypeptide comprising substitution of an amino acid corresponding to His-1083, Asp-1105 and/or Ser-1165, numbered relative to the full-length HCV-1 polyprotein such that protease activity is inhibited, and a core polypeptide;
(h) an E1 polypeptide, an E2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, and a core polypeptide;
(i) an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, and a core polypeptide; or
(j) an E1 polypeptide, an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, and a core polypeptide.

In yet a further embodiment, the invention is directed to a modified NS3 polypeptide comprising a substitution of an amino acid corresponding to His-1083, Asp-1105 and/or Ser-1165, numbered relative to the full-length HCV-1 polyprotein such that protease activity is inhibited when the modified NS3 polypeptide is present in an HCV fusion protein.

Yet another embodiment of the invention provides an isolated polynucleotide which encodes any of the proteins detailed above, recombinant vectors comprising the same, host cells transformed with the vectors, and methods of recombinantly producing the fusion proteins.

The invention also provides compositions comprising any of these fusion proteins, polynucleotides encoding the fusions, or recombinant vectors including the polynucleotides, and a pharmaceutically acceptable carrier.

Yet another embodiment of the invention provides a method of stimulating a cellular immune response in a vertebrate subject by administering a composition as described herein. In certain embodiments, the composition primes and/or activates T cells which recognize an epitope of an HCV polypeptide. T cells are contacted with a fusion protein comprising a modified NS3 polypeptide and at least one additional HCV polypeptide. A population of activated T cells recognizes an epitope of the NS3 and/or the additional HCV polypeptide(s).

The invention thus provides methods and reagents for stimulating a cellular immune response to HCV, such as for priming and/or activating T cells which recognize epitopes of HCV polypeptides. These methods and reagents are particularly advantageous for identifying epitopes of HCV polypeptides associated with a strong CTL response and for immunizing mammals, including humans, against HCV.

### BRIEF DESCRIPTION OF THE FIGURES

FIG. 1 is a diagrammatic representation of the HCV genome, depicting the various regions of the HCV polyprotein.

FIG. 2 depicts the DNA and corresponding amino acid sequence of a representative native, unmodified NS3 protease domain.

FIG. 3 shows the DNA and corresponding amino acid sequence of a representative modified fusion protein, with the NS3 protease domain deleted from the N-terminus and including amino acids 1-121 of Core on the C-terminus.

### DETAILED DESCRIPTION OF THE INVENTION

The practice of the present invention will employ, unless otherwise indicated, conventional methods of chemistry, biochemistry, recombinant DNA techniques and immunology, within the skill of the art. Such techniques are explained fully in the literature. See, e.g., Sambrook, et al., Molecular Cloning: A Laboratory Manual (2nd Edition); Methods In Enzymology (S. Colowick and N. Kaplan eds., Academic Press, Inc.); DNA Cloning, Vols. I and II (D. N. Glover ed.); Oligonucleotide Synthesis (M. J. Gait ed.); Nucleic Acid Hybridization (B. D. Hames & S. J. Higgins eds.); Animal Cell Culture (R. K. Freshney ed.); Perbal, B., A Practical Guide to Molecular Cloning.

All publications, patents and patent applications cited herein, whether supra or infra, are hereby incorporated by reference in their entirety.

It must be noted that, as used in this specification and the appended claims, the singular forms “a”, “an” and “the” include plural referents unless the content clearly dictates otherwise. Thus, for example, reference to “an antigen” includes a mixture of two or more antigens, and the like.

The following amino acid abbreviations are used throughout the text:

| | |
|---|---|
| Alanine: Ala (A) | Arginine: Arg (R) |
| Asparagine: Asn (N) | Aspartic acid: Asp (D) |
| Cysteine: Cys (C) | Glutamine: Gln (Q) |
| Glutamic acid: Glu (E) | Glycine: Gly (G) |
| Histidine: His (H) | Isoleucine: Ile (I) |
| Leucine: Leu (L) | Lysine: Lys (K) |
| Methionine: Met (M) | Phenylalanine: Phe (F) |
| Proline: Pro (P) | Serine: Ser (S) |
| Threonine: Thr (T) | Tryptophan: Trp (W) |
| Tyrosine: Tyr (Y) | Valine: Val (V) |

I. Definitions

In describing the present invention, the following terms will be employed, and are intended to be defined as indicated below.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification.

An HCV polypeptide is a polypeptide, as defined above, derived from the HCV polyprotein. The polypeptide need not be physically derived from HCV, but may be synthetically or recombinantly produced. Moreover, the polypeptide may be derived from any of the various HCV strains and isolates including isolates having any of the 6 genotypes of HCV described in Simmonds et al., J. Gen. Virol. (1993) 74:2391-2399 (e.g., strains 1, 2, 3, 4 etc.), as well as newly identified isolates, and subtypes of these isolates, such as HCV1a, HCV1b etc. A number of conserved and variable regions are known between these strains and, in general, the amino acid sequences of epitopes derived from these regions will have a high degree of sequence homology, e.g., amino acid sequence homology of more than 30%, preferably more than 40%, when the two sequences are aligned. Thus, for example, the term “NS4” polypeptide refers to native NS4 from any of the various HCV strains, as well as NS4 analogs, muteins and immunogenic fragments, as defined further below.

The terms “analog” and “mutein” refer to biologically active derivatives of the reference molecule, or fragments of such derivatives, that retain desired activity, such as the ability to stimulate a cell-mediated immune response, as defined below. In the case of a modified NS3, an “analog” or “mutein” refers to an NS3 molecule that lacks its native proteolytic activity. In general, the term “analog” refers to compounds having a native polypeptide sequence and structure with one or more amino acid additions, substitutions (generally conservative in nature, or in the case of modified NS3, non-conservative in nature at the active proteolytic site) and/or deletions, relative to the native molecule, so long as the modifications do not destroy immunogenic activity. The term “mutein” refers to peptides having one or more peptide mimics (“peptoids”), such as those described in International Publication No. WO 91/04282. Preferably, the analog or mutein has at least the same immunoactivity as the native molecule. Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

By “modified NS3” is meant an NS3 polypeptide with a modification such that protease activity of the NS3 polypeptide is disrupted. The modification can include one or more amino acid additions, substitutions (generally non-conservative in nature) and/or deletions, relative to the native molecule, wherein the protease activity of the NS3 polypeptide is disrupted. Methods of measuring protease activity are discussed further below.

By “fragment” is intended a polypeptide consisting of only a part of the intact full-length polypeptide sequence and structure. The fragment can include a C-terminal deletion and/or an N-terminal deletion of the native polypeptide. An “immunogenic fragment” of a particular HCV protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, that define an epitope, or any integer between 5 amino acids and the full-length sequence, provided that the fragment in question retains immunogenic activity, as measured by the assays described herein.

The term “epitope” as used herein refers to a sequence of at least about 3 to 5, preferably about 5 to 10 or 15, and not more than about 1,000 amino acids (or any integer therebetween), which define a sequence that by itself or as part of a larger sequence, binds to an antibody generated in response to such sequence. There is no critical upper limit to the length of the fragment, which may comprise nearly the full-length of the protein sequence, or even a fusion protein comprising two or more epitopes from the HCV polyprotein. An epitope for use in the subject invention is not limited to a polypeptide having the exact sequence of the portion of the parent protein from which it is derived. Indeed, viral genomes are in a state of constant flux and contain several variable domains which exhibit relatively high degrees of variability between isolates. Thus the term “epitope” encompasses sequences identical to the native sequence, as well as modifications to the native sequence, such as deletions, additions and substitutions (generally conservative in nature).

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., J. Mol. Biol. (1982) 157:105-132 for hydropathy plots.

For a description of various HCV epitopes, see, e.g., Chien et al., Proc. Natl. Acad. Sci. USA (1992) 89:10011-10015; Chien et al., J. Gastroent. Hepatol. (1993) 8:S33-39; Chien et al., International Publication No. WO 93/00365; Chien, D. Y., International Publication No. WO 94/01778; and U.S. Pat. Nos. 6,280,927 and 6,150,087, incorporated herein by reference in their entireties.

As used herein the term “T-cell epitope” refers to a feature of a peptide structure which is capable of inducing T-cell immunity towards the peptide structure or an associated hapten. T-cell epitopes generally comprise linear peptide determinants that assume extended conformations within the peptide-binding cleft of MHC molecules, (Unanue et al., Science (1987) 236:551-557). Conversion of polypeptides to MHC class II-associated linear peptide determinants (generally between 5-14 amino acids in length) is termed “antigen processing” which is carried out by antigen presenting cells (APCs). More particularly, a T-cell epitope is defined by local features of a short peptide structure, such as primary amino acid sequence properties involving charge and hydrophobicity, and certain types of secondary structure, such as helicity, that do not depend on the folding of the entire polypeptide. Further, it is believed that short peptides capable of recognition by helper T-cells are generally amphipathic structures comprising a hydrophobic side (for interaction with the MHC molecule) and a hydrophilic side (for interacting with the T-cell receptor), (Margalit et al., Computer Prediction of T-cell Epitopes, New Generation Vaccines Marcel-Dekker, Inc, ed. G. C. Woodrow et al., (1990) pp. 109-116) and further that the amphipathic structures have an α-helical configuration (see, e.g., Spouge et al., J. Immunol. (1987) 138:204-212; Berkower et al., J. Immunol. (1986) 136:2498-2503).

Hence, segments of proteins that include T-cell epitopes can be readily predicted using numerous computer programs. (See e.g., Margalit et al., Computer Prediction of T-cell Epitopes, New Generation Vaccines Marcel-Dekker, Inc, ed. G. C. Woodrow et al., (1990) pp. 109-116). Such programs generally compare the amino acid sequence of a peptide to sequences known to induce a T-cell response, and search for patterns of amino acids which are believed to be required for a T-cell epitope.

An “immunological response” to an HCV antigen (including both polypeptide and polynucleotides encoding polypeptides that are expressed in vivo) or composition is the development in a subject of a humoral and/or a cellular immune response to molecules present in the composition of interest. For purposes of the present invention, a “humoral immune response” refers to an immune response mediated by antibody molecules, while a “cellular immune response” is one mediated by T lymphocytes and/or other white blood cells. One important aspect of cellular immunity involves an antigen-specific response by cytolytic T cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Both CD8+ and CD4+ T cells are capable of killing HCV-infected cells. Another aspect of cellular immunity involves an antigen-specific response by helper T cells. Helper T cells act to help stimulate the function, and focus the activity of, nonspecific effector cells against cells displaying peptide antigens in association with MHC molecules on their surface. A “cellular immune response” also refers to the production of antiviral cytokines, chemokines and other such molecules produced by activated T cells and/or other white blood cells, including those derived from CD4+ and CD8+ T cells, including, but not limited to IFN-γ and TNF-α.

A composition or vaccine that elicits a cellular immune response may serve to sensitize a vertebrate subject by the presentation of antigen in association with MHC molecules at the cell surface. The cell-mediated immune response is directed at, or near, cells presenting antigen at their surface. In addition, antigen-specific T lymphocytes can be generated to allow for the future protection of an immunized host.

The ability of a particular antigen to stimulate a cell-mediated immunological response may be determined by a number of assays, such as by lymphoproliferation (lymphocyte activation) assays, CTL cytotoxic cell assays, or by assaying for T lymphocytes specific for the antigen in a sensitized subject. Such assays are well known in the art. See, e.g., Erickson et al., J. Immunol. (1993) 151:4189-4199; Doe et al., Eur. J. Immunol. (1994) 24:2369-2376; and the examples below.

Thus, an immunological response as used herein may be one which stimulates the production of CTLs, and/or the production or activation of helper T cells. The antigen of interest may also elicit an antibody-mediated immune response. Hence, an immunological response may include one or more of the following effects: the production of antibodies by B-cells; and/or the activation of suppressor T cells and/or γδ T cells directed specifically to an antigen or antigens present in the composition or vaccine of interest. These responses may serve to neutralize infectivity, and/or mediate antibody-complement, or antibody dependent cell cytotoxicity (ADCC) to provide protection or alleviation of symptoms to an immunized host. Such responses can be determined using standard immunoassays and neutralization assays, well known in the art.

By “equivalent antigenic determinant” is meant an antigenic determinant from different sub-species or strains of HCV, such as from strains 1, 2, 3, etc., of HCV which antigenic determinants are not necessarily identical due to sequence variation, but which occur in equivalent positions in the HCV sequence in question. In general the amino acid sequences of equivalent antigenic determinants will have a high degree of sequence homology, e.g., amino acid sequence homology of more than 30%, usually more than 40%, such as more than 60%, and even more than 80-90% homology, when the two sequences are aligned.

A “coding sequence” or a sequence which “encodes” a selected polypeptide, is a nucleic acid molecule which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

A “nucleic acid” molecule or “polynucleotide” can include both double- and single-stranded sequences and refers to, but is not limited to, cDNA from viral, procaryotic or eucaryotic mRNA, genomic DNA sequences from viral (e.g. DNA viruses and retroviruses) or procaryotic DNA, and especially synthetic DNA sequences. The term also captures sequences that include any of the known base analogs of DNA and RNA.

“Operably linked” refers to an arrangement of elements wherein the components so described are configured so as to perform their desired function. Thus, a given promoter operably linked to a coding sequence is capable of effecting the expression of the coding sequence when the proper transcription factors, etc., are present. The promoter need not be contiguous with the coding sequence, so long as it functions to direct the expression thereof. Thus, for example, intervening untranslated yet transcribed sequences can be present between the promoter sequence and the coding sequence, as can transcribed introns, and the promoter sequence can still be considered “operably linked” to the coding sequence.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

A “control element” refers to a polynucleotide sequence which aids in the expression of a coding sequence to which it is linked. The term includes promoters, transcription termination sequences, upstream regulatory domains, polyadenylation signals, untranslated regions, including 5′-UTRs and 3′-UTRs and when appropriate, leader sequences and enhancers, which collectively provide for the transcription and translation of a coding sequence in a host cell.

A “promoter” as used herein is a DNA regulatory region capable of binding RNA polymerase in a host cell and initiating transcription of a downstream (3′ direction) coding sequence operably linked thereto. For purposes of the present invention, a promoter sequence includes the minimum number of bases or elements necessary to initiate transcription of a gene of interest at levels detectable above background. Within the promoter sequence is a transcription initiation site, as well as protein binding domains (consensus sequences) responsible for the binding of RNA polymerase. Eucaryotic promoters will often, but not always, contain “TATA” boxes and “CAT” boxes.

A control sequence “directs the transcription” of a coding sequence in a cell when RNA polymerase will bind the promoter sequence and transcribe the coding sequence into mRNA, which is then translated into the polypeptide encoded by the coding sequence.

“Expression cassette” or “expression construct” refers to an assembly which is capable of directing the expression of the sequence(s) or gene(s) of interest. The expression cassette includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single-stranded DNA (e.g., a M13 origin of replication), at least one multiple cloning site, and a “mammalian” origin of replication (e.g., a SV40 or adenovirus origin of replication).

“Transformation,” as used herein, refers to the insertion of an exogenous polynucleotide into a host cell, irrespective of the method used for insertion: for example, transformation by direct uptake, transfection, infection, and the like. For particular methods of transfection, see further below. The exogenous polynucleotide may be maintained as a nonintegrated vector, for example, an episome, or alternatively, may be integrated into the host genome.

A “host cell” is a cell which has been transformed, or is capable of transformation, by an exogenous DNA sequence.

By “isolated” is meant, when referring to a polypeptide, that the indicated molecule is separate and discrete from the whole organism with which the molecule is found in nature or is present in the substantial absence of other biological macromolecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “purified” as used herein preferably means at least 75% by weight, more preferably at least 85% by weight, more preferably still at least 95% by weight, and most preferably at least 98% by weight, of biological macromolecules of the same type are present.

“Homology” refers to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98%, or more, sequence identity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

By “nucleic acid immunization” is meant the introduction of a nucleic acid molecule encoding one or more selected immunogens into a host cell, for the in vivo expression of the immunogen or immunogens. The nucleic acid molecule can be introduced directly into the recipient subject, such as by injection, inhalation, oral, intranasal and mucosal administration, or the like, or can be introduced ex vivo, into cells which have been removed from the host. In the latter case, the transformed cells are reintroduced into the subject where an immune response can be mounted against the antigen encoded by the nucleic acid molecule.

As used herein, “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

By “vertebrate subject” is meant any member of the subphylum cordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The invention described herein is intended for use in any of the above vertebrate species, since the immune systems of all of these vertebrates operate similarly.

II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of compositions and methods similar or equivalent to those described herein can be used in the practice of the present invention, the preferred materials and methods are described herein.

The present invention pertains to fusion proteins and polynucleotides encoding the same, comprising a modified NS3 polypeptide and at least one other HCV polypeptide from the HCV polyprotein. The fusion proteins of the present invention can be used to stimulate a cellular immune response, such as to activate HCV-specific T cells, i.e., T cells which recognize epitopes of these polypeptides and/or to elicit the production of helper T cells and/or to stimulate the production of antiviral cytokines, chemokines, and the like. Activation of HCV-specific T cells by such fusion proteins provides both in vitro and in vivo model systems for the development of HCV vaccines, particularly for identifying HCV polypeptide epitopes associated with a response. The fusion proteins can also be used to generate an immune response against HCV in a mammal, for example a CTL response, and/or to prime CD8+ and CD4+ T cells to produce antiviral agents, for either therapeutic or prophylactic purposes.

In order to further an understanding of the invention, a more detailed discussion is provided below regarding fusion proteins for use in the subject compositions, as well as production of the proteins, compositions comprising the same and methods of using the proteins.

Fusion Proteins

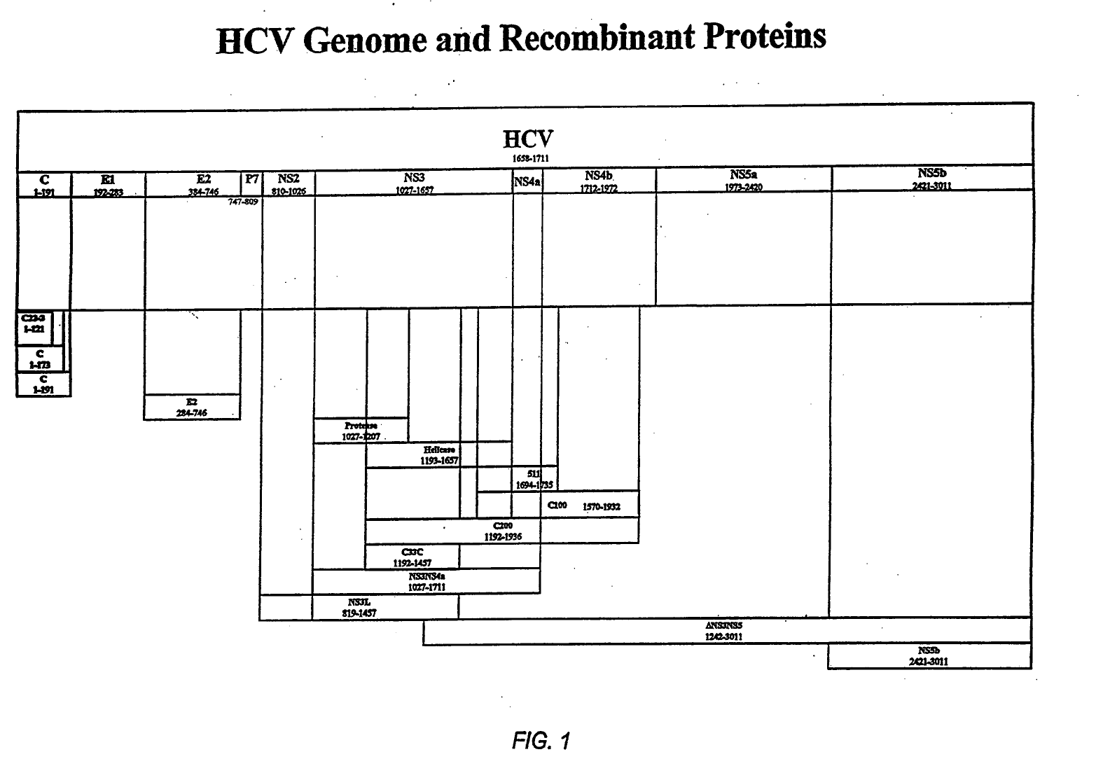

The genomes of HCV strains contain a single open reading frame of approximately 9,000 to 12,000 nucleotides, which is transcribed into a polyprotein. As shown in FIG. 1 and Table 1, an HCV polyprotein, upon cleavage, produces at least ten distinct products, in the order of NH₂₋Core-E1-E2-p7-NS2-NS3-NS4a-NS4b-NS5a-NS5b-COOH. The core polypeptide occurs at positions 1-191, numbered relative to HCV-1 (see, Choo et al. (1991) Proc. Natl. Acad. Sci. USA 88:2451-2455, for the HCV-1 genome). This polypeptide is further processed to produce an HCV polypeptide with approximately amino acids 1-173. The envelope polypeptides, E1 and E2, occur at about positions 192-383 and 384-746, respectively. The P7 domain is found at about positions 747-809. NS2 is an integral membrane protein with proteolytic activity and is found at about positions 810-1026 of the polyprotein. NS2, in combination with NS3, (found at about positions 1027-1657), cleaves the NS2-NS3 sissle bond which in turn generates the NS3 N-terminus and releases a large polyprotein that includes both serine protease and RNA helicase activities. The NS3 protease, found at about positions 1027-1207, serves to process the remaining polyprotein. The helicase activity is found at about positions 1193-1657. NS3 liberates an NS3 cofactor (NS4a, found about positions 1658-1711), two proteins (NS4b found at about positions 1712-1972, and NS5a found at about positions 1973-2420), and an RNA-dependent RNA polymerase (NS5b found at about positions 2421-3011).

Completion of polyprotein maturation is initiated by autocatalytic cleavage at the NS3-Ns4a junction, catalyzed by the NS3 serine protease.

TABLE 1

| Domain | Approximate Boundaries* |
|---|---|
| C (core) | 1-191 |
| E1 | 192-383 |
| E2 | 384-746 |
| P7 | 747-809 |
| NS2 | 810-1026 |
| NS3 | 1027-1657 |
| NS4a | 1658-1711 |
| NS4b | 1712-1972 |
| NS5a | 1973-2420 |
| NS5b | 2421-3011 |

*Numbered relative to HCV-1. See, Choo et al. (1991) Proc. Natl. Acad. Sci. USA 88: 2451-2455.

Fusion proteins of the invention include an NS3 polypeptide modified to inhibit protease activity, such that further cleavage of the fusion is inhibited. The NS3 polypeptide can be modified by deletion of all or a portion of the NS3 protease domain. Alternatively, proteolytic activity can be inhibited by substitutions of amino acids within active regions of the protease domain. Finally, additions of amino acids to active regions of the domain, such that the catalytic site is modified, will also serve to inhibit proteolytic activity.

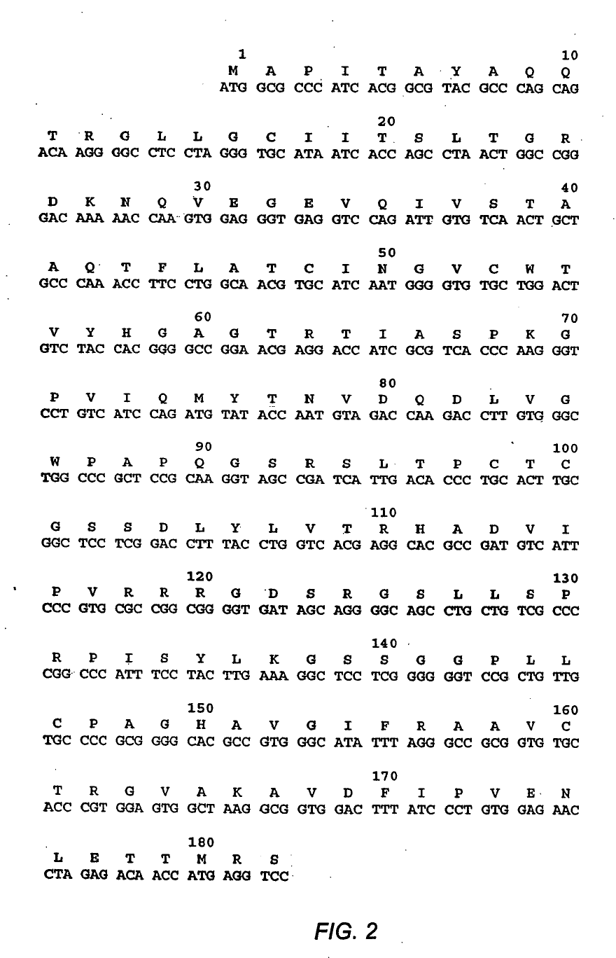

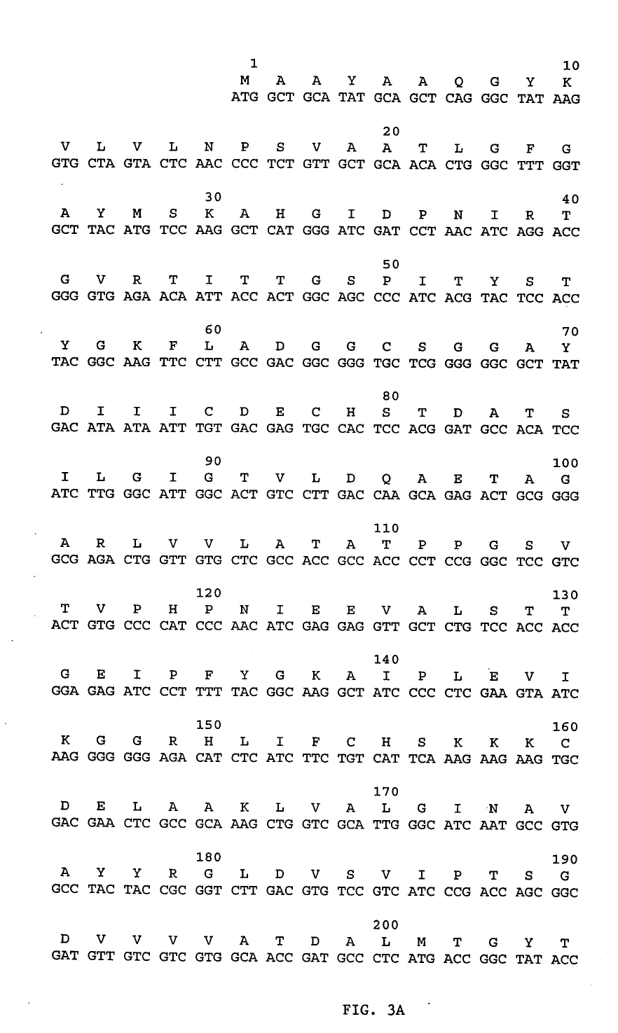

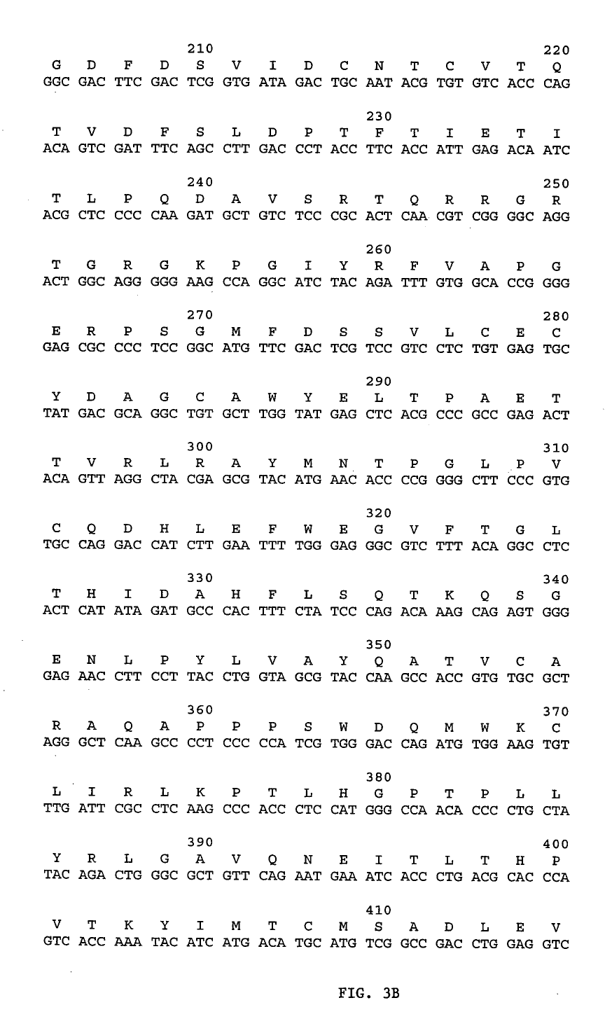

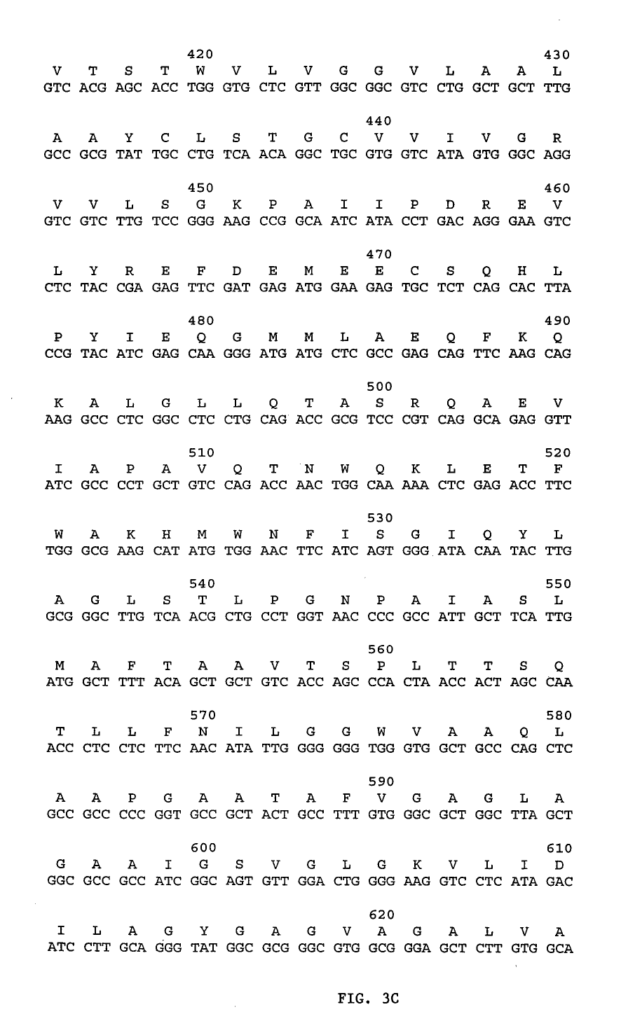

As explained above, the protease activity is found at about amino acid positions 1027-1207, numbered relative to the full-length HCV-1 polyprotein (see, Choo et al., Proc. Natl. Acad. Sci. USA (1991) 88:2451-2455), positions 2-182 of FIG. 2. The structure of the NS3 protease and active site are known. See, e.g., De Francesco et al., Antivir. Ther. (1998) 3:99-109; Koch et al., Biochemistry (2001) 40:631-640. Thus, deletions or modifications to the native sequence will typically occur at or near the active site of the molecule. Particularly, it is desirable to modify or make deletions to one or more amino acids occurring at positions 1- or 2-182, preferably 1- or 2-170, or 1- or 2-155 of FIG. 2. Preferred modifications are to the catalytic triad at the active site of the protease, i.e., H, D and/or S residues, in order to inactivate the protease. These residues occur at positions 1083, 1105 and 1165, respectively, numbered relative to the full-length HCV polyprotein (positions 58, 80 and 140, respectively, of FIG. 2). Such modifications will suppress proteolytic cleavage while maintaining T-cell epitopes. One of skill in the art can readily determine portions of the NS3 protease to delete in order to disrupt activity. The presence or absence of activity can be determined using methods known to those of skill in the art.

For example, protease activity or lack thereof may be determined using the procedure described below in the examples, as well as using assays well known in the art. See, e.g., Takeshita et al., Anal. Biochem. (1997) 247:242-246; Kakiuchi et al., J. Biochem. (1997) 122:749-755; Sali et al., Biochemistry (1998) 37:3392-3401; Cho et al., J. Virol. Meth. (1998) 72:109-115; Cerretani et al., Anal. Biochem. (1999) 266:192-197; Zhang et al., Anal. Biochem. (1999) 270:268-275; Kakiuchi et al., J. Virol. Meth. (1999) 80:77-84; Fowler et al., J. Biomol. Screen. (2000) 5:153-158; and Kim et al., Anal. Biochem. (2000) 284:42-48.

The fusion protein of the present invention includes, in addition to the modified NS3 polypeptide, one or more polypeptides from one or more other regions of an HCV polyprotein. In fact, the fusion can include all the regions of the HCV polyprotein. These polypeptides may derived from the same HCV isolate as the NS3 polypeptide, or from different strains and isolates including isolates having any of the various HCV genotypes, to provide increased protection against a broad range of HCV genotypes. Additionally, polypeptides can be selected based on the particular viral clades endemic in specific geographic regions where vaccine compositions containing the fusions will be used. It is readily apparent that the subject fusions provide an effective means of treating HCV infection in a wide variety of contexts.

In certain embodiments, the fusion protein comprises a modified NS3 (also referred to herein as NS3*), an NS4 (NS4a and NS4b), an NS5a and, optionally, a core polypeptide of an HCV (NS3*NS4NS5a or NS3*NS4NS5aCore fusion proteins, also termed “NS3*45a” and “NS3*45aCore” herein). These regions need not be in the order in which they naturally occur in the native HCV polyprotein. Thus, for example, the core polypeptide may be at the N- and/or C-terminus of the fusion.

Another embodiment provides a fusion protein that includes an NS3*, an NS4, an NS5a, an NS5b, and optionally, a core polypeptide of an HCV (NS3*NS4NS5aNS5b or NS3*NS4NS5aNS5bCore fusion proteins, also termed “NS3*45ab” and “NS3*45abCore” herein). These regions need not be in the order in which they naturally occur in the native HCV polyprotein. Thus, for example, the core polypeptide may be at the N- and/or C-terminus of the fusion.

Yet other embodiments are directed to a fusion protein comprising an NS3* combined with an NS2, an NS3* combined with an NS2, p7 and E2, an NS3* combined with an NS2, p7 and an E1, an NS3* combined with an NS2, p7 and an E1 and an E2, an NS3* combined with an E2, an NS3* combined with an E1 and an E2, all with or without a core polypeptide. As with those fusions described above, these regions need not be in the order in which they occur naturally. Moreover, each of these regions can be derived from the same or a different HCV isolate.

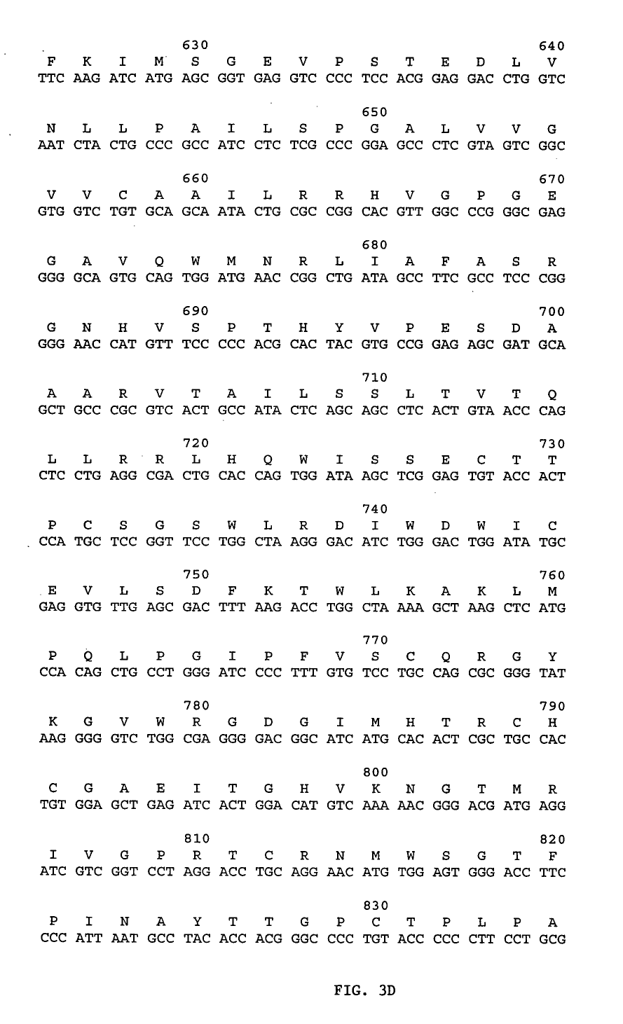

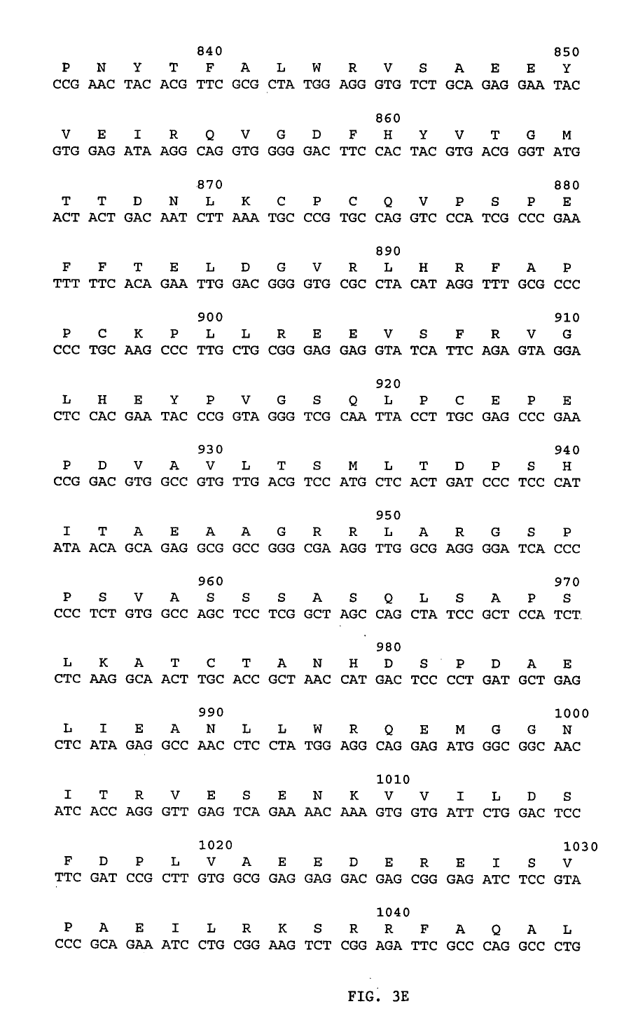

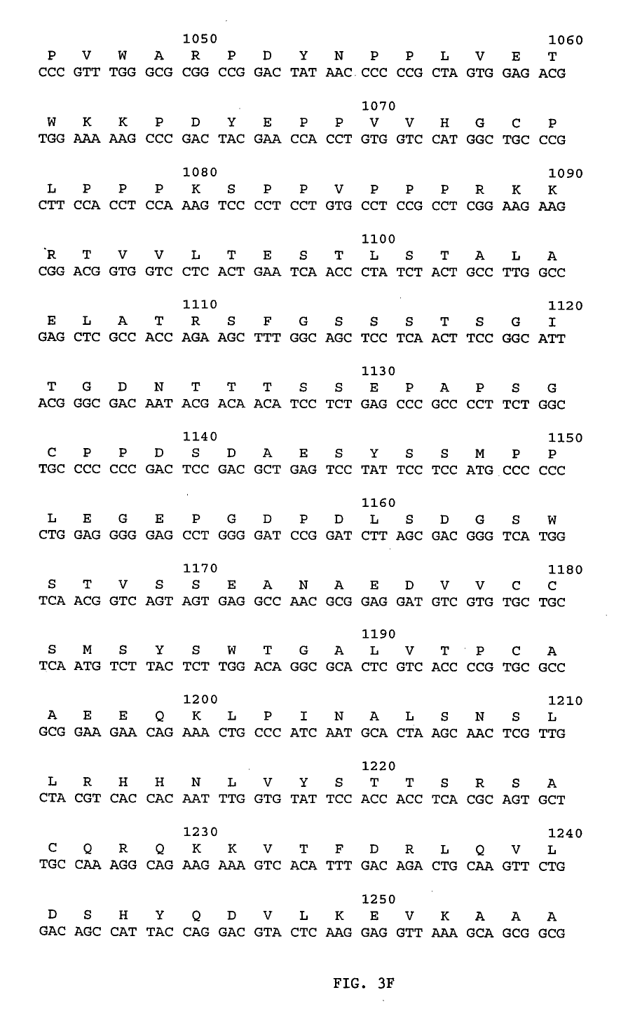

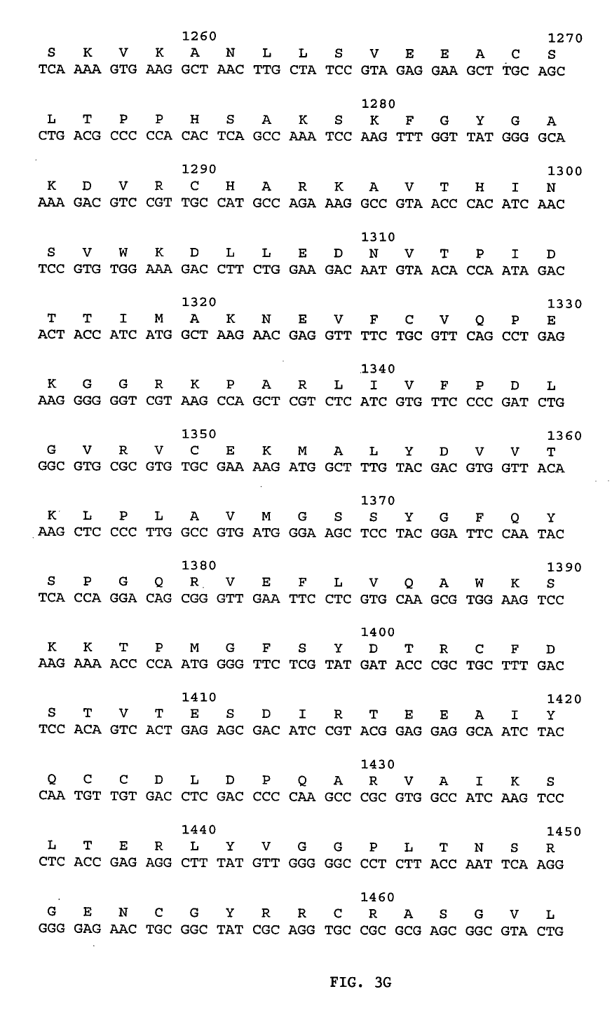

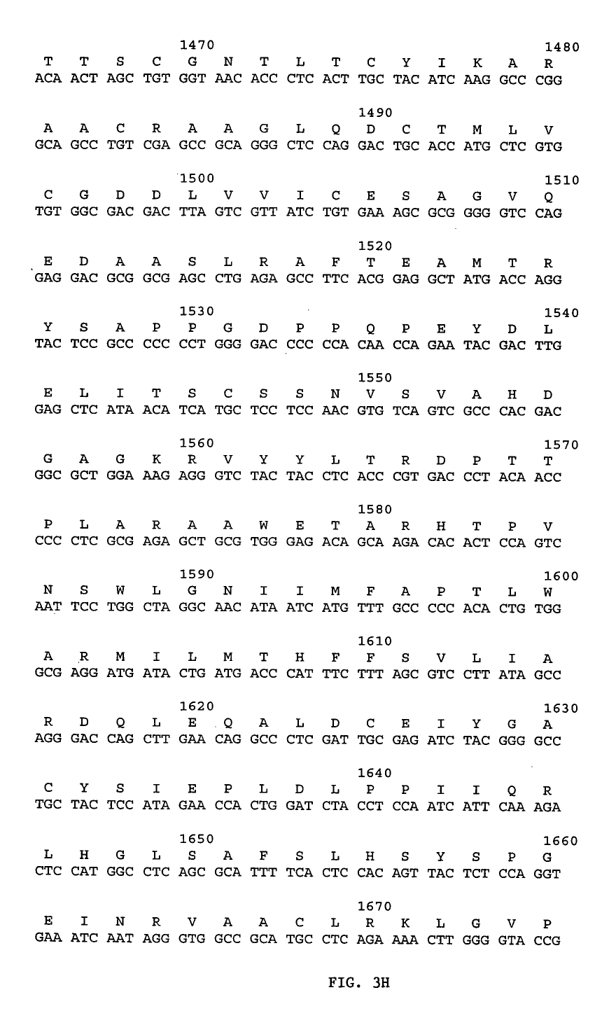

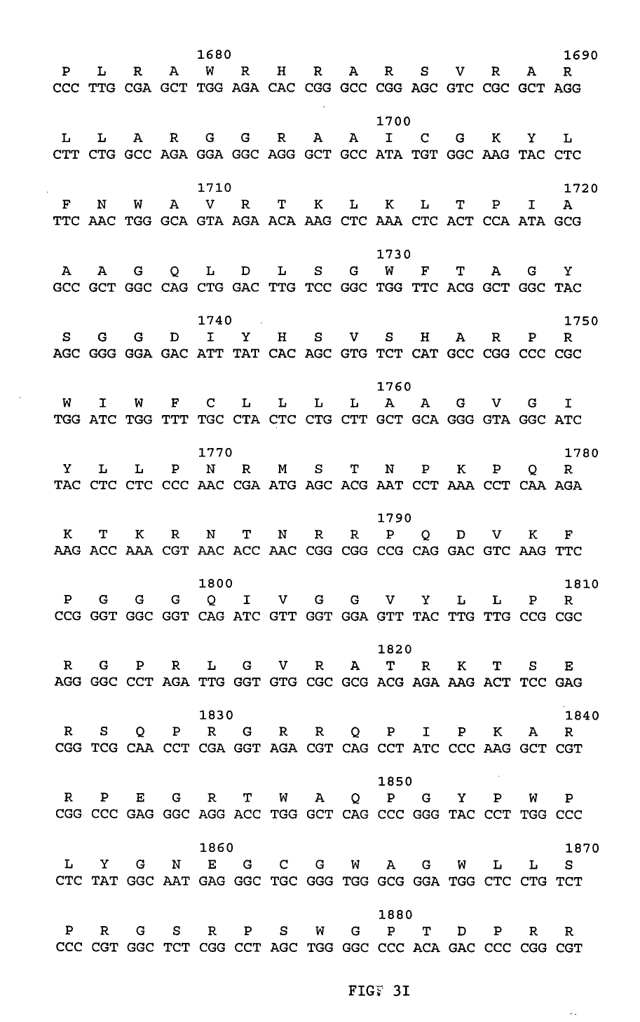

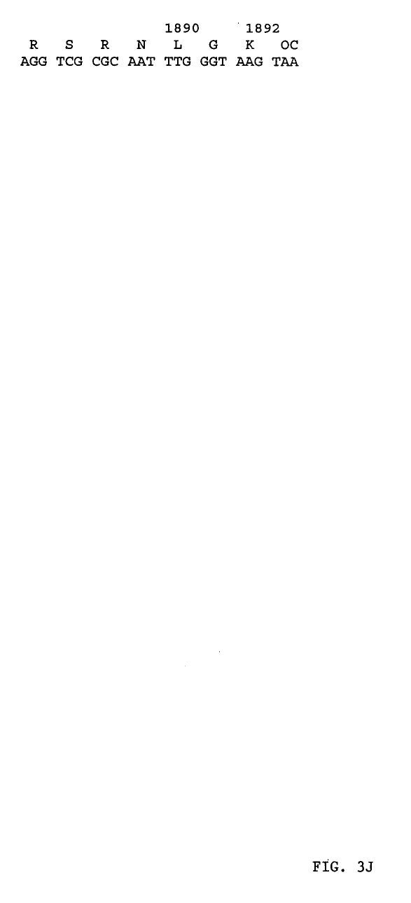

FIG. 3 shows a representative modified fusion protein, with the NS3 protease domain deleted from the N-terminus and including amino acids 1-121 of Core on the C-terminus.

The various HCV polypeptides present in the various fusions described above can either be full-length polypeptides or portions thereof. The portions of the HCV polypeptides making up the fusion protein comprise at least one epitope, which is recognized by a T cell receptor on an activated T cell, such as 2152-HEYPVGSQL-2160 (SEQ ID NO:1) and/or 2224-AELIEANLLWRQEMG-2238 (SEQ ID NO:2). Epitopes of NS2, p7, E1, E2, NS3, NS4 (NS4a and NS4b), NS5a, NS5b, NS3NS4NS5a, and NS3NS4NS5aNS5b can be identified by several methods. For example, the individual polypeptides or fusion proteins comprising any combination of the above, can be isolated, by, e.g., immunoaffinity purification using a monoclonal antibody for the polypeptide or protein. The isolated protein sequence can then be screened by preparing a series of short peptides by proteolytic cleavage of the purified protein, which together span the entire protein sequence. By starting with, for example, 100-mer polypeptides, each polypeptide can be tested for the presence of epitopes recognized by a T-cell receptor on an HCV-activated T cell, progressively smaller and overlapping fragments can then be tested from an identified 100-mer to map the epitope of interest.

Epitopes recognized by a T-cell receptor on an HCV-activated T cell can be identified by, for example, ⁵¹Cr release assay (see Example 4) or by lymphoproliferation assay (see Example 6). In a ⁵¹Cr release assay, target cells can be constructed that display the epitope of interest by cloning a polynucleotide encoding the epitope into an expression vector and transforming the expression vector into the target cells. HCV-specific CD8⁺ T cells will lyse target cells displaying, for example, one or more epitopes from one or more regions of the HCV polyprotein found in the fusion, and will not lyse cells that do not display such an epitope. In a lymphoproliferation assay, HCV-activated CD4⁺ T cells will proliferate when cultured with, for example, one or more epitopes from one or more regions of the HCV polyprotein found in the fusion, but not in the absence of an HCV epitopic peptide.

The various HCV polypeptides can occur in any order in the fusion protein. If desired, at least 2, 3, 4, 5, 6, 7, 8, 9, or 10 or more of one or more of the polypeptides may occur in the fusion protein. Multiple viral strains of HCV occur, and HCV polypeptides of any of these strains can be used in a fusion protein.

Nucleic acid and amino acid sequences of a number of HCV strains and isolates, including nucleic acid and amino acid sequences of the various regions of the HCV polyprotein, including Core, NS2, p7, E1, E2, NS3, NS4, NS5a, NS5b genes and polypeptides have been determined. For example, isolate HCV J1.1 is described in Kubo et al. (1989) Japan. Nucl. Acids Res. 17:10367-10372; Takeuchi et al.(1990) Gene 91:287-291; Takeuchi et al. (1990) J. Gen. Virol. 71:3027-3033; and Takeuchi et al. (1990) Nucl. Acids Res. 18:4626. The complete coding sequences of two independent isolates, HCV-J and BK, are described by Kato et al., (1990) Proc. Natl. Acad. Sci. USA 87:9524-9528 and Takamizawa et al., (1991) J. Virol. 65:1105-1113 respectively.

Publications that describe HCV-1 isolates include Choo et al. (1990) Brit. Med. Bull. 46:423-441; Choo et al. (1991) Proc. Natl. Acad. Sci. USA 88:2451-2455 and Han et al. (1991) Proc. Natl. Acad. Sci. USA 88:1711-1715. HCV isolates HC-J1 and HC-J4 are described in Okamoto et al. (1991) Japan J. Exp. Med. 60:167-177. HCV isolates HCT 18˜, HCT 23, Th, HCT 27, EC1 and EC10 are described in Weiner et al. (1991) Virol. 180:842-848. HCV isolates Pt-1, HCV-K1 and HCV-K2 are described in Enomoto et al. (1990) Biochem. Biophys. Res. Commun. 170:1021-1025. HCV isolates A, C, D & E are described in Tsukiyama-Kohara et al. (1991) Virus Genes 5:243-254.

Each of the components of a fusion protein can be obtained from the same HCV strain or isolate or from different HCV strains or isolates. Fusion proteins comprising HCV polypeptides from, for example, the NS3 polypeptide can be derived from a first strain of HCV, and the other HCV polypeptides present can be derived from a second strain of HCV. Alternatively, one or more of the other HCV polypeptides, for example NS2, NS4, Core, p7, E1 and/or E2, if present, can be derived from a first strain of HCV, and the remaining HCV polypeptides can be derived from a second strain of HCV. Additionally, each or the HCV polypeptides present can be derived from different HCV strains.

As explained above, it may be desirable to include polypeptides derived from the core region of the HCV polyprotein in the fusions of the invention. This region occurs at amino acid positions 1-191 of the HCV polyprotein, numbered relative to HCV-1. Either the full-length protein, fragments thereof, such as amino acids 1-160, e.g., amino acids 1-150, 1-140, 1-130, 1-120, for example, amino acids 1-121, 1-122, 1-123 . . . 1-151, etc., or smaller fragments containing epitopes of the full-length protein may be used in the subject fusions, such as those epitopes found between amino acids 10-53, amino acids 10-45, amino acids 67-88, amino acids 120-130, or any of the core epitopes identified in, e.g., Houghton et al., U.S. Pat. No. 5,350,671; Chien et al., Proc. Natl. Acad. Sci. USA (1992) 89:10011-10015; Chien et al., J. Gastroent. Hepatol. (1993) 8:S33-39; Chien et al., International Publication No. WO 93/00365; Chien, D. Y., International Publication No. WO 94/01778; and U.S. Pat. Nos. 6,280,927 and 6,150,087, the disclosures of which are incorporated herein by reference in their entireties. Moreover, a protein resulting from a frameshift in the core region of the polyprotein, such as described in International Publication No. WO 99/63941, may be used.

If a core polypeptide is present, it can occur at the N- terminus, the C-terminus and/or internal to the fusion. Particularly preferred is a core polypeptide on the C-terminus as this allows for the formation of complexes with certain adjuvants, such as ISCOMs, described further below.

As described above, useful polypeptides in the HCV fusion include T-cell epitopes derived from any of the various regions in the polyprotein. In this regard, E1, E2, p7 and NS2 are known to contain human T-cell epitopes (both CD4+ and CD8+) and including one or more of these epitopes serves to increase vaccine efficacy as well as to increase protective levels against multiple HCV genotypes. Moreover, multiple copies of specific, conserved T-cell epitopes can also be used in the fusions, such as a composite of epitopes from different genotypes.

For example, polypeptides from the HCV E1 and/or E2 regions can be used in the fusions of the present invention. E2 exists as multiple species (Spaete et al., Virol. (1992) 188:819-830; Selby et al., J. Virol. (1996) 70:5177-5182; Grakoui et al., J. Virol. (1993) 67:1385-1395; Tomei et al., J. Virol. (1993) 67:4017-4026) and clipping and proteolysis may occur at the N- and C-termini of the E2 polypeptide. Thus, an E2 polypeptide for use herein may comprise amino acids 405-661, e.g., 400, 401, 402 . . . to 661, such as 383 or 384-661, 383 or 384-715, 383 or 384-746, 383 or 384-749 or 383 or 384-809, or 383 or 384 to any C-terminus between 661-809, of an HCV polyprotein, numbered relative to the full-length HCV-1 polyprotein. Similarly, E1 polypeptides for use herein can comprise amino acids 192-326, 192-330, 192-333, 192-360, 192-363, 192-383, or 192 to any C-terminus between 326-383, of an HCV polyprotein.

Immunogenic fragments of E1 and/or E2 which comprise epitopes may be used in the subject fusions. For example, fragments of E1 polypeptides can comprise from about to nearly the full-length of the molecule, such as 6, 10, 25, 50, 75, 100, 125, 150, 175, 185 or more amino acids of an E1 polypeptide, or any integer between the stated numbers. Similarly, fragments of E2 polypeptides can comprise 6, 10, 25, 50, 75, 100, 150, 200, 250, 300, or 350 amino acids of an E2 polypeptide, or any integer between the stated numbers.

For example, epitopes derived from, e.g., the hypervariable region of E2, such as a region spanning amino acids 384-410 or 390-410, can be included in the fusions. A particularly effective E2 epitope to incorporate into an E2 polypeptide sequence is one which includes a consensus sequence derived from this region, such as the consensus sequence

Gly-Ser-Ala-Ala-Arg-Thr-Thr-Ser-Gly-Phe-Val-Ser-Leu-Phe-Ala-Pro-Gly-Ala-Lys-Gln-Asn, which represents a consensus sequence for amino acids 390-410 of the HCV type 1 genome. Additional epitopes of E1 and E2 are known and described in, e.g., Chien et al., International Publication No. WO 93/00365.

Moreover, the E1 and/or E2 polypeptides may lack all or a portion of the membrane spanning domain. With E1, generally polypeptides terminating with about amino acid position 370 and higher (based on the numbering of HCV-1 E1) will be retained by the ER and hence not secreted into growth media. With E2, polypeptides terminating with about amino acid position 731 and higher (also based on the numbering of the HCV-1 E2 sequence) will be retained by the ER and not secreted. (See, e.g., International Publication No. WO 96/04301, published Feb. 15, 1996). It should be noted that these amino acid positions are not absolute and may vary to some degree. Thus, the present invention contemplates the use of E1 and/or E2 polypeptides which retain the transmembrane binding domain, as well as polypeptides which lack all or a portion of the transmembrane binding domain, including E1 polypeptides terminating at about amino acids 369 and lower, and E2 polypeptides, terminating at about amino acids 730 and lower. Furthermore, the C-terminal truncation can extend beyond the transmembrane spanning domain towards the N-terminus. Thus, for example, E1 truncations occurring at positions lower than, e.g., 360 and E2 truncations occurring at positions lower than, e.g., 715, are also encompassed by the present invention. All that is necessary is that the truncated E1 and E2 polypeptides remain functional for their intended purpose. However, particularly preferred truncated E1 constructs are those that do not extend beyond about amino acid 300. Most preferred are those terminating at position 360. Preferred truncated E2 constructs are those with C-terminal truncations that do not extend beyond about amino acid position 715. Particularly preferred E2 truncations are those molecules truncated after any of amino acids 715-730, such as 725.

For a description of various HCV epitopes from these and other HCV regions, see, e.g., Chien et al., Proc. Natl. Acad. Sci. USA (1992) 89:10011-10015; Chien et al., J. Gastroent. Hepatol. (1993) 8:S33-39; Chien et al., International Publication No. WO 93/00365; Chien, D. Y., International Publication No. WO 94/01778; and U.S. Pat. Nos. 6,280,927 and 6,150,087, incorporated herein by reference in their entireties.

Preferably, the above-described fusion proteins, as well as the individual components of these proteins, are produced recombinantly. A polynucleotide encoding these proteins can be introduced into an expression vector which can be expressed in a suitable expression system. A variety of bacterial, yeast, mammalian and insect expression systems are available in the art and any such expression system can be used. Optionally, a polynucleotide encoding these proteins can be translated in a cell-free translation system. Such methods are well known in the art. The proteins also can be constructed by solid phase protein synthesis.

If desired, the fusion proteins, or the individual components of these proteins, also can contain other amino acid sequences, such as amino acid linkers or signal sequences, as well as ligands useful in protein purification, such as glutathione-S-transferase and staphylococcal protein A.

Polynucleotides Encoding the Fusion Proteins

Polynucleotides contain less than an entire HCV genome, or alternatively can include the sequence of the entire polyprotein with a mutated NS3 domain, as described above. The polynucleotides can be RNA or single- or double-stranded DNA. Preferably, the polynucleotides are isolated free of other components, such as proteins and lipids. The polynucleotides encode the fusion proteins described above, and thus comprise coding sequences for NS3* and at least one other HCV polypeptide from a different region of the HCV polyprotein, such as polypeptides derived from NS2, p7, E1, E2, NS4, NS5a, NS5b, core, etc. Polynucleotides of the invention can also comprise other nucleotide sequences, such as sequences coding for linkers, signal sequences, or ligands useful in protein purification such as glutathione-S-transferase and staphylococcal protein A.

To aid expression yields, it may be desirable to split the polyprotein into fragments for expression. These fragments can be used in combination in compositions as described herein. Alternatively, these fragments can be joined subsequent to expression. Thus, for example, NS3*NS4Core can be expressed as one construct and NS5aNS5bCore can be expressed as a second construct. Similarly, NS3*NS4NS5a can be expressed as one construct and a second construct with, e.g., NS3*NS4NS5aCore can be expressed as a second construct. For example, NS2p7E2NS3*NS4 can be expressed as a single construct, and NS3(unmodified)NS4NS5b can be expressed as an additional construct for use in the subject compositions. It is to be understood that the above combinations are merely representative and any combination of fusions can be expressed separately.

Polynucleotides encoding the various HCV polypeptides can be isolated from a genomic library derived from nucleic acid sequences present in, for example, the plasma, serum, or liver homogenate of an HCV infected individual or can be synthesized in the laboratory, for example, using an automatic synthesizer. An amplification method such as PCR can be used to amplify polynucleotides from either HCV genomic DNA or cDNA encoding therefor.

Polynucleotides can comprise coding sequences for these polypeptides which occur naturally or can be artificial sequences which do not occur in nature. These polynucleotides can be ligated to form a coding sequence for the fusion proteins using standard molecular biology techniques. If desired, polynucleotides can be cloned into an expression vector and transformed into, for example, bacterial, yeast, insect, or mammalian cells so that the fusion proteins of the invention can be expressed in and isolated from a cell culture.

The expression constructs of the present invention, including the desired fusion, or individual expression constructs comprising the individual components of these fusions, may be used for nucleic acid immunization, to stimulate a cellular immune response, using standard gene delivery protocols. Methods for gene delivery are known in the art. See, e.g., U.S. Pat. Nos. 5,399,346, 5,580,859, 5,589,466, incorporated by reference herein in their entireties. Genes can be delivered either directly to the vertebrate subject or, alternatively, delivered ex vivo, to cells derived from the subject and the cells reimplanted in the subject. For example, the constructs can be delivered as plasmid DNA, e.g., contained within a plasmid, such as pBR322, pUC, or ColE1 Additionally, the expression constructs can be packaged in liposomes prior to delivery to the cells. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991) 1097:1-17; Straubinger et al., in Methods of Enzymology (1983), Vol. 101, pp. 512-527.

Liposomal preparations for use with the present invention include cationic (positively charged), anionic (negatively charged) and neutral preparations, with cationic liposomes particularly preferred. Cationic liposomes are readily available. For example, N[1-2,3-dioleyloxy)propyl]-N,N,N-triethyl-ammonium (DOTMA) liposomes are available under the trademark Lipofectin, from GIBCO BRL, Grand Island, N.Y. (See, also, Felgner et al., Proc. Natl. Acad. Sci. USA (1987) 84:7413-7416). Other commercially available lipids include transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes. The various liposome-nucleic acid complexes are prepared using methods known in the art. See, e.g., Straubinger et al., in METHODS OF IMMUNOLOGY (1983), Vol. 101, pp. 512-527; Szoka et al., Proc. Natl. Acad. Sci. USA (1978) 75:4194-4198; Papahadjopoulos et al., Biochim. Biophys. Acta (1975) 394:483; Wilson et al., Cell (1979) 17:77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); Enoch and Strittmatter, Proc. Natl. Acad. Sci. USA (1979) 76:145); Fraley et al., J. Biol. Chem. (1980) 255:10431; Szoka and Papahadjopoulos, Proc. Natl. Acad. Sci. USA (1978) 75:145; and Schaefer-Ridder et al., Science (1982) 215:166.

The DNA can also be delivered in cochleate lipid compositions similar to those described by Papahadjopoulos et al., Biochem. Biophys. Acta. (1975) 394:483-491. See, also, U.S. Pat. Nos. 4,663,161 and 4,871,488.

A number of viral based systems have been developed for gene transfer into mammalian cells. For example, retroviruses provide a convenient platform for gene delivery systems, such as murine sarcoma virus, mouse mammary tumor virus, Moloney murine leukemia virus, and Rous sarcoma virus. A selected gene can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman, BioTechniques (1989) 7:980-990; Miller, A. D., Human Gene Therapy (1990) 1:5-14; Scarpa et al., Virology (1991) 180:849-852; Burns et al., Proc. Natl. Acad. Sci. USA (1993) 90:8033-8037; and Boris-Lawrie and Temin, Cur. Opin. Genet. Develop. (1993) 3:102-109. Briefly, retroviral gene delivery vehicles of the present invention may be readily constructed from a wide variety of retroviruses, including for example, B, C, and D type retroviruses as well as spumaviruses and lentiviruses such as FIV, HIV, HIV-1, HIV-2 and SIV (see RNA Tumor Viruses, Second Edition, Cold Spring Harbor Laboratory, 1985). Such retroviruses may be readily obtained from depositories or collections such as the American Type Culture Collection (“ATCC”; 10801 University Blvd., Manassas, Va. 20110-2209), or isolated from known sources using commonly available techniques.

A number of adenovirus vectors have also been described, such as adenovirus Type 2 and Type 5 vectors. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476).

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as but not limited to vectors derived from the Sindbis and Semliki Forest viruses, VEE, will also find use as viral vectors for delivering the gene of interest. For a description of Sindbis-virus derived vectors useful for the practice of the instant methods, see, Dubensky et al., J. Virol. (1996) 70:508-519; and International Publication Nos. WO 95/07995 and WO 96/17072.

Other vectors can be used, including but not limited to simian virus 40 and cytomegalovirus. Bacterial vectors, such as Salmonella ssp. Yersinia enterocolitica, Shigella spp., Vibrio cholerae, Mycobacterium strain BCG, and Listeria monocytogenes can be used. Minichromosomes such as MC and MC 1, bacteriophages, cosmids (plasmids into which phage lambda cos sites have been inserted) and replicons (genetic elements that are capable of replication under their own control in a cell) can also be used.

The expression constructs may also be encapsulated, adsorbed to, or associated with, particulate carriers. Such carriers present multiple copies of a selected molecule to the immune system and promote trapping and retention of molecules in local lymph nodes. The particles can be phagocytosed by macrophages and can enhance antigen presentation through cytokine release. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; and McGee et al., J. Microencap. (1996).

A wide variety of other methods can be used to deliver the expression constructs to cells. Such methods include DEAE dextran-mediated transfection, calcium phosphate precipitation, polylysine- or polyornithine-mediated transfection, or precipitation using other insoluble inorganic salts, such as strontium phosphate, aluminum silicates including bentonite and kaolin, chromic oxide, magnesium silicate, talc, and the like. Other useful methods of transfection include electroporation, sonoporation, protoplast fusion, liposomes, peptoid delivery, or microinjection. See, e.g., Sambrook et al., supra, for a discussion of techniques for transforming cells of interest; and Felgner, P. L., Advanced Drug Delivery Reviews (1990) 5:163-187, for a review of delivery systems useful for gene transfer. One particularly effective method of delivering DNA using electroporation is described in International Publication No. WO/0045823.

Additionally, biolistic delivery systems employing particulate carriers such as gold and tungsten, are especially useful for delivering the expression constructs of the present invention. The particles are coated with the construct to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744.

Compositions Comprising Fusion Proteins or Polynucleotides

The invention also provides compositions comprising the fusion proteins or polynucleotides. The compositions may include one or more fusions, so long as one of the fusions includes a mutated NS3 domain as described herein. Compositions of the invention may also comprise a pharmaceutically acceptable carrier. The carrier should not itself induce the production of antibodies harmful to the host. Pharmaceutically acceptable carriers are well known to those in the art. Such carriers include, but are not limited to, large, slowly metabolized, macromolecules, such as proteins, polysaccharides such as latex functionalized sepharose, agarose, cellulose, cellulose beads and the like, polylactic acids, polyglycolic acids, polymeric amino acids such as polyglutamic acid, polylysine, and the like, amino acid copolymers, and inactive virus particles.

Pharmaceutically acceptable salts can also be used in compositions of the invention, for example, mineral salts such as hydrochlorides, hydrobromides, phosphates, or sulfates, as well as salts of organic acids such as acetates, proprionates, malonates, or benzoates. Especially useful protein substrates are serum albumins, keyhole limpet hemocyanin, immunoglobulin molecules, thyroglobulin, ovalbumin, tetanus toxoid, and other proteins well known to those of skill in the art. Compositions of the invention can also contain liquids or excipients, such as water, saline, glycerol, dextrose, ethanol, or the like, singly or in combination, as well as substances such as wetting agents, emulsifying agents, or pH buffering agents. The proteins of the invention can also be adsorbed to, entrapped within or otherwise associated with liposomes and particulate carriers such as PLG. Liposomes and other particulate carriers are described above.

If desired, co-stimulatory molecules which improve immunogen presentation to lymphocytes, such as B7-1 or B7-2, or cytokines, lymphokines, and chemokines, including but not limited to cytokines such as IL-2, modified IL-2 (cys125 ser125), GM-CSF, IL-12, γ-interferon, IP-10, MIP1β, FLP-3, ribavirin and RANTES, may be included in the composition. Optionally, adjuvants can also be included in a composition. Adjuvants which can be used include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59 (PCT Publ. No. WO 90/14837), containing 5% Squalene, 0.5% Tween 80, and 0.5% Span 85 (optionally containing various amounts of MTP-PE), formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL +CWS (Detox™); (3) saponin adjuvants, such as QS21 or Stimulon™ (Cambridge Bioscience, Worcester, Mass.) may be used or particles generated therefrom such as ISCOMs (immunostimulating complexes), which ISCOMs may be devoid of additional detergent (see, e.g., International Publication No. WO 00/07621); (4) Complete Freunds Adjuvant (CFA) and Incomplete Freunds Adjuvant (IFA); (5) cytokines, such as interleukins, such as IL-1, IL-2, IL-4, IL-5, IL-6, IL-7, IL-12 etc. (see, e.g., International Publication No. WO 99/44636), interferons, such as gamma interferon, macrophage colony stimulating factor (M-CSF), tumor necrosis factor (TNF), etc.; (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., International Publication Nos. WO93/13202 and WO092/19265); (7) monophosporyl lipid A (MPL) or 3-O-deacylated MPL (3dMPL) (see, e.g., GB 2220221; EPA 0689454), optionally in the substantial absence of alum (see, e.g., International Publication No. WO 00/56358); (8) combinations of 3dMPL with, for example, QS21 and/or oil-in-water emulations (see, e.g., EPA 0835318; EPA 0735898; EPA 0761231); (9) a polyoxyethylene ether or a polyoxyethylene ester (see, e.g., International Publication No. WO 99/52549); (10) an immunostimulatory oligonucleotide such as a CpG oligonucleotide, or a saponin and an immunostimulatory oligonucleotide, such as a CpG oligonucleotide (see, e.g., International Publication No. WO 00/62800); (11) an immunostimulant and a particle of a metal salt (see, e.g., International Publication No. WO 00/23105); (12) a saponin and an oil-in-water emulsion (see, e.g., International Publication No. WO 99/11241; (13) a saponin (e.g., QS21)+3dMPL+IL-12 (optionally +a sterol) (see, e.g., International Publication No. WO 98/57659); (14) the MPL derivative RC529; and (15) other substances that act as immunostimulating agents to enhance the effectiveness of the composition. Alum and MF59 are preferred.

As mentioned above, muramyl peptides include, but are not limited to, N-acetyl-muramyl-L-threonyl-D-isoglutamine (thr-MDP), -acetyl-normuramyl-L-alanyl-D-isoglutamine (CGP 11637, referred to nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), etc.

Moreover, the fusion protein can be adsorbed to, or entrapped within, an ISCOM. Classic ISCOMs are formed by combination of cholesterol, saponin, phospholipid, and immunogens, such as viral envelope proteins. Generally, immunogens (usually with a hydrophobic region) are solubilized in detergent and added to the reaction mixture, whereby ISCOMs are formed with the immunogen incorporated therein. ISCOM matrix compositions are formed identically, but without viral proteins. Proteins with high positive charge may be electrostatically bound in the ISCOM particles, rather than through hydrophobic forces. For a more detailed general discussion of saponins and ISCOMs, and methods of formulating ISCOMs, see Barr et al. (1998) Adv. Drug Delivery Reviews 32:247-271 (1998).

ISCOMs for use with the present invention are produced using standard techniques, well known in the art, and are described in e.g., U.S. Pat. Nos. 4,981,684, 5,178,860, 5,679,354 and 6,027,732; European Publ. Nos. EPA 109,942; 180,564 and 231,039; Coulter et al. (1998) Vaccine 16:1243. Typically, the term “ISCOM” refers to immunogenic complexes formed between glycosides, such as triterpenoid saponins (particularly Quil A), and antigens which contain a hydrophobic region. See, e.g., European Publ. Nos. EPA 109,942 and 180,564. In this embodiment, the HCV fusions (usually with a hydrophobic region) are solubilized in detergent and added to the reaction mixture, whereby ISCOMs are formed with the fusions incorporated therein. The HCV polypeptide ISCOMs are readily made with HCV polypeptides which show amphipathic properties. However, proteins and peptides which lack the desirable hydrophobic properties may be incorporated into the immunogenic complexes after coupling with peptides having hydrophobic amino acids, fatty acid radicals, alkyl radicals and the like.

As explained in European Publ. No. EPA 231,039, the presence of antigen is not necessary in order to form the basic ISCOM structure (referred to as a matrix or ISCOMATRIX), which may be formed from a sterol, such as cholesterol, a phospholipid, such as phosphatidylethanolamine, and a glycoside, such as Quil A. Thus, the HCV fusion of interest, rather than being incorporated into the matrix, is present on the outside of the matrix, for example adsorbed to the matrix via electrostatic interactions. For example, HCV fusions with high positive charge may be electrostatically bound to the ISCOM particles, rather than through hydrophobic forces. For a more detailed general discussion of saponins and ISCOMs, and methods of formulating ISCOMs, see Barr et al. (1998) Adv. Drug Delivery Reviews 32:247-271 (1998).

The ISCOM matrix may be prepared, for example, by mixing together solubilized sterol, glycoside and (optionally) phospholipid. If phospholipids are not used, two dimensional structures are formed. See, e.g., European Publ. No. EPA 231,039. The term “ISCOM matrix” is used to refer to both the 3-dimensional and 2-dimensional structures. The glycosides to be used are generally glycosides which display amphipathic properties and comprise hydrophobic and hydrophilic regions in the molecule. Preferably saponins are used, such as the saponin extract from Quillaja saponaria Molina and Quil A. Other preferred saponins are aescine from Aesculus hippocastanum (Patt et al. (1960) Arzneimittelforschung 10:273-275 and sapoalbin from Gypsophilla struthium (Vochten et al. (1968) J. Pharm. Belg. 42:213-226.

In order to prepare the ISCOMs, glycosides are used in at least a critical micelle-forming concentration. In the case of Quil A, this concentration is about 0.03% by weight. The sterols used to produce ISCOMs may be known sterols of animal or vegetable origin, such as cholesterol, lanosterol, lumisterol, stigmasterol and sitosterol. Suitable phospholipids include phosphatidylcholine and phosphatidylethanolamine. Generally, the molar ratio of glycoside (especially when it is Quil A) to sterol (especially when it is cholesterol) to phospholipid is 1:1:0-1, ±20% (preferably not more than ±10%) for each figure. This is equivalent to a weight ratio of about 5:1 for the Quil A:cholesterol.

A solubilizing agent may also be present and may be, for example a detergent, urea or guanidine. Generally, a non-ionic, ionic or zwitter-ionic detergent or a cholic acid based detergent, such as sodium desoxycholate, cholate and CTAB (cetyltriammonium bromide), can be used for this purpose. Examples of suitable detergents include, but are not limited to, octylglucoside, nonyl N-methyl glucamide or decanoyl N-methyl glucamide, alkylphenyl polyoxyethylene ethers such as a polyethylene glycol p-isooctyl-phenylether having 9 to 10 oxyethylene groups (commercialized under the trade name TRITON X-100R™), acylpolyoxyethylene esters such as acylpolyoxyethylene sorbitane esters (commercialized under the trade name TWEEN 20™, TWEEN 80™, and the like). The solubilizing agent is generally removed for formation of the ISCOMs, such as by ultrafiltration, dialysis, ultracentrifugation or chromatography, however, in certain methods, this step is unnecessary. (See, e.g., U.S. Pat. No. 4,981,684).

Generally, the ratio of glycoside, such as QuilA, to HCV fusion by weight is in the range of 5:1 to 0.5:1. Preferably the ratio by weight is approximately 3:1 to 1:1, and more preferably the ratio is 2:1.

Once the ISCOMs are formed, they may be formulated into compositions and administered to animals, as described herein. If desired, the solutions of the immunogenic complexes obtained may be lyophilized and then reconstituted before use.

Methods of Producing HCV-Specific Antibodies

The HCV fusion proteins can be used to produce HCV-specific polyclonal and monoclonal antibodies. HCV-specific polyclonal and monoclonal antibodies specifically bind to HCV antigens. Polyclonal antibodies can be produced by administering the fusion protein to a mammal, such as a mouse, a rabbit, a goat, or a horse. Serum from the immunized animal is collected and the antibodies are purified from the plasma by, for example, precipitation with ammonium sulfate, followed by chromatography, preferably affinity chromatography. Techniques for producing and processing polyclonal antisera are known in the art.

Monoclonal antibodies directed against HCV-specific epitopes present in the fusion proteins can also be readily produced. Normal B cells from a mammal, such as a mouse, immunized with an HCV fusion protein, can be fused with, for example, HAT-sensitive mouse myeloma cells to produce hybridomas. Hybridomas producing HCV-specific antibodies can be identified using RIA or ELISA and isolated by cloning in semi-solid agar or by limiting dilution. Clones producing HCV-specific antibodies are isolated by another round of screening.

Antibodies, either monoclonal and polyclonal, which are directed against HCV epitopes, are particularly useful for detecting the presence of HCV or HCV antigens in a sample, such as a serum sample from an HCV-infected human. An immunoassay for an HCV antigen may utilize one antibody or several antibodies. An immunoassay for an HCV antigen may use, for example, a monoclonal antibody directed towards an HCV epitope, a combination of monoclonal antibodies directed towards epitopes of one HCV polypeptide, monoclonal antibodies directed towards epitopes of different HCV polypeptides, polyclonal antibodies directed towards the same HCV antigen, polyclonal antibodies directed towards different HCV antigens, or a combination of monoclonal and polyclonal antibodies. Immunoassay protocols may be based, for example, upon competition, direct reaction, or sandwich type assays using, for example, labeled antibody. The labels may be, for example, fluorescent, chemiluminescent, or radioactive.

The polyclonal or monoclonal antibodies may further be used to isolate HCV particles or antigens by immunoaffinity columns. The antibodies can be affixed to a solid support by, for example, adsorption or by covalent linkage so that the antibodies retain their immunoselective activity. Optionally, spacer groups may be included so that the antigen binding site of the antibody remains accessible. The immobilized antibodies can then be used to bind HCV particles or antigens from a biological sample, such as blood or plasma. The bound HCV particles or antigens are recovered from the column matrix by, for example, a change in pH.

HCV-Specific T Cells

HCV-specific T cells that are activated by the above-described fusions, including the NS3*NS4NS5a fusion protein or NS3*NS4NS5aNS5b fusion protein, with or without a core polypeptide, as well as any of the other various fusions described herein, expressed in vivo or in vitro, preferably recognize an epitope of an HCV polypeptide such as an NS2, p7, E1, E2, NS3, NS4, NS5a or NSb polypeptide, including an epitope of a fusion of one or more of these peptides with an NS3*, with or without a core polypeptide. HCV-specific T cells can be CD8⁺ or CD4⁺.

HCV-specific CD8⁺ T cells can be cytotoxic T lymphocytes (CTL) which can kill HCV-infected cells that display any of these epitopes complexed with an MHC class I molecule. HCV-specific CD8⁺ T cells can be detected by, for example, ⁵¹Cr release assays (see Example 4). ⁵¹Cr release assays measure the ability of HCV-specific CD8⁺ T cells to lyse target cells displaying one or more of these epitopes. HCV-specific CD8⁺ T cells which express antiviral agents, such as IFN-γ, are also contemplated herein and can also be detected by immunological methods, preferably by intracellular staining for IFN-γ or like cytokine after in vitro stimulation with one or more of the HCV polypeptides, such as but not limited to an NS3, an NS4, an NS5a, or an NS5b polypeptide (see Example 5).

HCV-specific CD4⁺ cells activated by the above-described fusions, such as but not limited to an NS3*NS4NS5a or NS3*NS4NS5aNS5b fusion protein, with or without a core polypeptide, expressed in vivo or in vitro, preferably recognize an epitope of an HCV polypeptide, such as but not limited to an NS2, p7, E1, E2, NS3, NS4, NS5a, or NS5b polypeptide, including an epitope of fusions thereof, such as but not limited to an NS3NS4NS5a or NS3NS4NS5aNS5b fusion protein, that is bound to an MHC class II molecule on an HCV-infected cell and proliferate in response to stimulating, e.g., NS3*NS4NS5a or NS3*NS4NS5aNS5b peptides, with or without a core polypeptide.

HCV-specific CD4⁺ T cells can be detected by a lymphoproliferation assay (see Example 6). Lymphoproliferation assays measure the ability of HCV-specific CD4⁺ T cells to proliferate in response to, e.g., an NS2, p7, E1, E2, NS3, an NS4, an NS5a, and/or an NS5b epitope.

Methods of Activating HCV-Specific T Cells.

The HCV fusion proteins or polynucleotides can be used to activate HCV-specific T cells either in vitro or in vivo. Activation of HCV-specific T cells can be used, inter alia, to provide model systems to optimize CTL responses to HCV and to provide prophylactic or therapeutic treatment against HCV infection. For in vitro activation, proteins are preferably supplied to T cells via a plasmid or a viral vector, such as an adenovirus vector, as described above.

Polyclonal populations of T cells can be derived from the blood, and preferably from peripheral lymphoid organs, such as lymph nodes, spleen, or thymus, of mammals that have been infected with an HCV. Preferred mammals include mice, chimpanzees, baboons, and humans. The HCV serves to expand the number of activated HCV-specific T cells in the mammal. The HCV-specific T cells derived from the mammal can then be restimulated in vitro by adding, an HCV fusion protein as described herein, such as but not limited to HCV NS3NS4NS5a or NS3NS4NS5aNS5b epitopic peptides, with or without a core polypeptide, to the T cells. The HCV-specific T cells can then be tested for, inter alia, proliferation, the production of IFN-γ, and the ability to lyse target cells displaying, for example, NS3NS4NS5a or NS3NS4NS5aNS5b epitopes in vitro.

In a lymphoproliferation assay (see Example 6), HCV-activated CD4⁺ T cells proliferate when cultured with an HCV polypeptide, such as but not limited to an NS3, NS4, NS5a, NS5b, NS3NS4NS5a, or NS3NS4NS5aNS5b epitopic peptide, but not in the absence of an epitopic peptide. Thus, particular HCV epitopes, such as NS2, p7, E1, E2, NS3, NS4, NS5a, NS5b, and fusions of these epitopes, such as but not limited to NS3NS4NS5a and NS3NS4NS5aNS5b epitopes that are recognized by HCV-specific CD4⁺ T cells can be identified using a lymphoproliferation assay.

Similarly, detection of IFN-γ in HCV-specific CD4⁺ and/or CD8⁺ T cells after in vitro stimulation with the above-described fusion proteins, can be used to identify, for example, fusion protein epitopes, such as but not limited to NS2, p7, E1, E2, NS3, NS4, NS5a, NS5b, and fusions of these epitopes, such as but not limited to NS3NS4NS5a, and NS3NS4NS5aNS5b epitopes that are particularly effective at stimulating CD4⁺ and/or CD8⁺ T cells to produce IFN-γ (see Example 5).

Further, ⁵¹Cr release assays are useful for determining the level of CTL response to HCV. See Cooper et al. Immunity 10:439-449. For example, HCV-specific CD8⁺ T cells can be derived from the liver of an HCV infected mammal. These T cells can be tested in ⁵¹Cr release assays against target cells displaying, e.g., NS3NS4NS5a NS3NS4NS5aNS5b epitopes. Several target cell populations expressing different NS3NS4NS5a or NS3NS4NS5aNS5b epitopes can be constructed so that each target cell population displays different epitopes of NS3NS4NS5a or NS3NS4NS5aNS5b. The HCV-specific CD8⁺ cells can be assayed against each of these target cell populations. The results of the ⁵¹Cr release assays can be used to determine which epitopes of NS3NS4NS5a or NS3NS4NS5aNS5b are responsible for the strongest CTL response to HCV. NS3*NS4NS5a fusion proteins or NS3*NS4NS5aNS5b fusion proteins, with or without core polypeptides, which contain the epitopes responsible for the strongest CTL response can then be constructed using the information derived from the ⁵¹Cr release assays.

An HCV fusion protein as described above, or polynucleotide encoding such a fusion protein, can be administered to a mammal, such as a mouse, baboon, chimpanzee, or human, to activate HCV-specific T cells in vivo. Administration can be by any means known in the art, including parenteral, intranasal, intramuscular or subcutaneous injection, including injection using a biological ballistic gun (“gene gun”), as discussed above.

Preferably, injection of an HCV polynucleotide is used to activate T cells. In addition to the practical advantages of simplicity of construction and modification, injection of the polynucleotides results in the synthesis of a fusion protein in the host. Thus, these immunogens are presented to the host immune system with native post-translational modifications, structure, and conformation. The polynucleotides are preferably injected intramuscularly to a large mammal, such as a human, at a dose of 0.5, 0.75, 1.0, 1.5, 2.0, 2.5, 5 or 10 mg/kg.

A composition of the invention comprising an HCV fusion protein or polynucleotide is administered in a manner compatible with the particular composition used and in an amount which is effective to activate HCV-specific T cells as measured by, inter alia, a ⁵¹Cr release assay, a lymphoproliferation assay, or by intracellular staining for IFN-γ. The proteins and/or polynucleotides can be administered either to a mammal which is not infected with an HCV or can be administered to an HCV-infected mammal. The particular dosages of the polynucleotides or fusion proteins in a composition will depend on many factors including, but not limited to the species, age, and general condition of the mammal to which the composition is administered, and the mode of administration of the composition. An effective amount of the composition of the invention can be readily determined using only routine experimentation. In vitro and in vivo models described above can be employed to identify appropriate doses. The amount of polynucleotide used in the example described below provides general guidance which can be used to optimize the activation of HCV-specific T cells either in vivo or in vitro. Generally, 0.5, 0.75, 1.0, 1.5, 2.0, 2.5, 5 or 10 mg of an HCV fusion protein or polynucleotide, with or without a core polypeptide, will be administered to a large mammal, such as a baboon, chimpanzee, or human. If desired, co-stimulatory molecules or adjuvants can also be provided before, after, or together with the compositions.

Immune responses of the mammal generated by the delivery of a composition of the invention, including activation of HCV-specific T cells, can be enhanced by varying the dosage, route of administration, or boosting regimens. Compositions of the invention may be given in a single dose schedule, or preferably in a multiple dose schedule in which a primary course of vaccination includes 1-10 separate doses, followed by other doses given at subsequent time intervals required to maintain and/or reinforce an immune response, for example, at 1-4 months for a second dose, and if needed, a subsequent dose or doses after several months.

III. Experimental

Below are examples of specific embodiments for carrying out the present invention. The examples are offered for illustrative purposes only, and are not intended to limit the scope of the present invention in any way. Those of skill in the art will readily appreciate that the invention may be practiced in a variety of ways given the teaching of this disclosure.

Efforts have been made to ensure accuracy with respect to numbers used (e.g., amounts, temperatures, etc.), but some experimental error and deviation should, of course, be allowed for.

### EXAMPLE 1

### Production of NS3*NS4NS5aCore Polynucleotides

NS3* in the following examples represents a modified NS3 molecule. A polynucleotide encoding NS3NS4NS5a (approximately amino acids 1027 to 2399, numbered relative to HCV-1) (also termed “NS345a” herein) is isolated from an HCV. The NS3 portion of the molecule is mutagenzied by mutating the coding sequence for the His, Asp and Ser residues found at the protease active site, such that the resulting molecule codes for amino acids other than His, Asp and Ser at these positions and lacks NS3 protease activity. This construct is fused with a polynucleotide encoding a core polypeptide which includes amino acids 1-122 of the full-length polyprotein The core-encoding polynucleotide sequence is fused downstream from the NS5a-encoding portion of the construct such that the resulting fusion protein includes the core polypeptide at its C-terminus. The construct is cloned into plasmid, vaccinia virus, and adenovirus vectors. Additionally, the construct is inserted into a recombinant expression vector and used to transform host cells to produce the NS3*NS4NS5aCore fusion protein.

Protease enzyme activity is determined as follows. An NS4A peptide (KKGSVVIVGRIVLSGKPAIIPKK), and the fusion protein are diluted in 90 μl of reaction buffer (25 mM Tris, pH 7.5, 0.15M NaCl, 0.5 mM EDTA, 10% glycerol, 0.05 n-Dodecyl B-D-Maltoside, 5 mM DTT) and allowed to mix for 30 minutes at room temperature. 90 μl of the mixture is added to a microtiter plate (Costar, Inc., Corning, N.Y.) and 10 μl of HCV substrate (AnaSpec, Inc., San Jose Calif.) is added. The plate is mixed and read on a Fluostar plate reader. Results are expressed as relative fluorescence units (RFU) per minute.

### EXAMPLE 2

### Priming of HCV-Specific CTLs in Vaccinated Animals

The HCV fusion protein, NS3*NS4NS5aCore, produced as described above is used to produce an HCV fusion-ISCOM as follows. The fusion-ISCOM formulations are prepared by mixing the fusion protein with a preformed ISCOMATRIX (empty ISCOMs) utilizing ionic interactions to maximize association between the antigen and the adjuvant. ISCOMATRIX is prepared essentially as described in Coulter et al. (1998) Vaccine 16:1243.

Rhesus macaques are immunized under anesthesia. Animals are divided into two groups. The first group is infected with 2×10⁸ plaque forming units (pfu) (1×10⁸ intradermally and 1×10⁸ by scarification) of rVVC/E1 at month 0. This group serves as a positive control for CTL priming. Animals from the second group are immunized with 25-100 μg of an HCV fusion polypeptide, as described above, that has been adsorbed to an ISCOM, by intramuscular (IM) injection in the left quadriceps at months 0, 1, 2 and 6. Cytotoxic activity is assayed in a standard ⁵¹Cr release assay as described in, e.g., Paliard et al. (2000) AIDS Res. Hum. Retroviruses 16:273.

### EXAMPLE 3

### Immunization With NS3*NS4NS5aCore Polynucleotides

In one immunization protocol, animals are immunized with 50-250 μg of plasmid DNA encoding an NS3*NS4NS5aCore fusion protein by intramuscular injection into the tibialis anterior. A booster injection of 10⁷ pfu of vaccinia virus (VV)-NS5a (intraperitoneal) or 50-250 μg of plasmid control (intramuscular) is provided 6 weeks later.

In another immunization protocol, animals are injected intramuscularly in the tibialis anterior with 10¹⁰ adenovirus particles encoding an NS3*NS4NS5aCore fusion protein. An intraperitoneal booster injection of 10⁷ pfu of VV-NS5a or an intramuscular booster injection of 10¹⁰ adenovirus particles encoding NS3*NS4NS5aCore is provided 6 weeks later.

### EXAMPLE 4

### Activation of HCV-Specific CD8⁺ T Cells

⁵¹Cr Release Assay. A ⁵¹Cr release assay is used to measure the ability of HCV-specific T cells to lyse target cells displaying an NS5a epitope. Spleen cells are pooled from the immunized animals. These cells are restimulated in vitro for 6 days with the CTL epitopic peptide p214K9 (2152-HEYPVGSQL-2160; SEQ ID NO:1) from HCV-NS5a in the presence of IL-2. The spleen cells are then assayed for cytotoxic activity in a standard ⁵¹Cr release assay against peptide-sensitized target cells (L929) expressing class I, but not class II MHC molecules, as described in Weiss (1980) J. Biol. Chem. 255:9912-9917. Ratios of effector (T cells) to target (B cells) of 60:1, 20:1, and 7:1 are tested. Percent specific lysis is calculated for each effector to target ratio.

### EXAMPLE 5

### Activation of HCV-Specific CD8⁺ T Cells Which Express IFN-γ

Intracellular Staining for Interferon-gamma (IFN-γ). Intracellular staining for IFN-γ is used to identify the CD8⁺ T cells that secrete IFN-γ after in vitro stimulation with the NS5a epitope p214K9. Spleen cells of individual immunized animals are restimulated in vitro either with p214K9 or with a non-specific peptide for 6-12 hours in the presence of IL-2 and monensin. The cells are then stained for surface CD8 and for intracellular IFN-γ and analyzed by flow cytometry. The percent of CD8⁺ cells which are also positive for IFN-γ is then calculated.

### EXAMPLE 6

### Proliferation of HCV-Specific CD4⁺ T Cells

Lymphoproliferation assay. Spleen cells from pooled immunized animals are depleted of CD8⁺ T cells using magnetic beads and are cultured in triplicate with either p222D, an NS5a-epitopic peptide from HCV-NS5a (2224-AELIEANLLWRQEMG-2238; SEQ ID NO:2), or in medium alone. After 72 hours, cells are pulsed with 1 μCi per well of ³H-thymidine and harvested 6-8 hours later. Incorporation of radioactivity is measured after harvesting. The mean cpm is calculated.

### EXAMPLE 7

### Ability of NS3*45aCore-Encoding DNA Vaccine Formulations to Prime CTLs

Animals are immunized with either 10-250 μg of plasmid DNA encoding NS3*45aCore fusion protein as described in Example 3, with PLG-linked DNA encoding NS3*45aCore (see below), or with DNA encoding NS3*45aCore, delivered via electroporation (see, e.g., International Publication No. WO/0045823 for this delivery technique). The immunizations are followed by a booster injection 6 weeks later of plasmid DNA encoding NS3*45aCore.

PLG-delivered DNA. The polylactide-co-glycolide (PLG) polymers are obtained from Boehringer Ingelheim, U.S.A. The PLG polymer is RG505, which has a copolymer ratio of 50/50 and a molecular weight of 65 kDa (manufacturers data). Cationic microparticles with adsorbed DNA are prepared using a modified solvent evaporation process, essentially as described in Singh et al., Proc. Natl. Acad. Sci. USA (2000) 97:811-816. Briefly, the microparticles are prepared by emulsifying 10 ml of a 5% w/v polymer solution in methylene chloride with 1 ml of PBS at high speed using an IKA homogenizer. The primary emulsion is then added to 50 ml of distilled water containing cetyl trimethyl ammonium bromide (CTAB) (0.5% w/v). This results in the formation of a w/o/w emulsion which is stirred at 6000 rpm for 12 hours at room temperature, allowing the methylene chloride to evaporate. The resulting microparticles are washed twice in distilled water by centrifugation at 10,000 g and freeze dried. Following preparation, washing and collection, DNA is adsorbed onto the microparticles by incubating 100 mg of cationic microparticles in a 1 mg/ml solution of DNA at 4 C for 6 hours. The microparticles are then separated by centrifugation, the pellet washed with TE buffer and the microparticles are freeze dried.

CTL activity and IFN-γ expression is measured by ⁵¹Cr release assay or intracellular staining as described in the examples above.

### EXAMPLE 8

### Immunization Routes and Replicon Particles SINCR (DC+) Encoding for NS3*45aCore

Alphavirus replicon particles, for example, SINCR (DC+) are prepared as described in Polo et al., Proc. Natl. Acad. Sci. USA (1999) 96:4598-4603. Animals are injected with 5×10⁶ IU SINCR (DC+) replicon particles encoding for NS3*45aCore intramuscularly (IM) as described in Example 3, or subcutaneously (S/C) at the base of the tail (BoT) and foot pad (FP), or with a combination of ⅔ of the DNA delivered via IM administration and ⅓ via a BoT route. The immunizations are followed by a booster injection of vaccinia virus encoding NS5a as described in Example 3. IFN-γ expression is measured by intracellular staining as described in Example 5.

### EXAMPLE 9

### Alphavirus Replicon Priming, Followed by Various Boosting Regimes

Alphavirus replicon particles, for example, SINCR (DC+) are prepared as described in Polo et al., Proc. Natl. Acad. Sci. USA (1999) 96:4598-4603. Animals are primed with SINCR (DC+), 1.5×10⁶ IU replicon particles encoding NS345a, by intramuscular injection into the tibialis anterior, followed by a booster of either 10-100 μg of plasmid DNA encoding for NS5a, 10¹⁰ adenovirus particles encoding NS3*45aCore, 1.5×10⁶ IU SINCR (DC+) replicon particles encoding NS3*45aCore, or 10⁷ pfu vaccinia virus encoding NS5a at 6 weeks. IFN-γ expression is measured by intracellular staining as described in Example 5.

### EXAMPLE 10

### Alphaviruses Expressing NS3*45aCore

Alphavirus replicon particles, for example, SINCR (DC+) and SINCR (LP) are prepared as described in Polo et al., Proc. Natl. Acad. Sci. USA (1999) 96:4598-4603. Animals are immunized with 1×10² to 1×10⁶ IU SINCR (DC+) replicons encoding NS3*45aCore via a combination of delivery routes (⅔ IM and ⅓ S/C) as well as by S/C alone, or with 1×10² to 1×10⁶ IU SINCR (LP) replicon particles encoding NS3*45aCore via a combination of delivery routes (⅔ IM and ⅓ S/C) as well as by S/C alone. The immunizations are followed by a booster injection of 10⁷ pfu vaccinia virus encoding NS5a at 6 weeks. IFN-γ expression is measured by intracellular staining as described in Example 5.

Thus, HCV fusion polypeptides, to stimulate cell-mediated immune responses, are disclosed. Although preferred embodiments of the subject invention have been described in some detail, it is understood that obvious variations can be made without departing from the spirit and the scope of the invention as defined herein.

1. An immunogenic fusion protein comprising (a) a modified NS3 polypeptide comprising at least one amino acid substitution to the HCV NS3 region, such that protease activity is inhibited, and (b) at least one polypeptide derived from a region of the HCV polyprotein other than the NS3 region.

2. The fusion protein of claim 1, wherein the modification comprises a substitution of an amino acid corresponding to His-1083, Asp-1105 and/or Ser-1165, numbered relative to the full-length HCV-1 polyprotein.

3. The fusion protein of claim 1, wherein the protein comprises a modified NS3 polypeptide, an NS4 polypeptide, an NS5a polypeptide, and optionally a core polypeptide.

4. The fusion protein of claim 3, wherein the protein further comprises an NS5b polypeptide, and optionally a core polypeptide.

5. The fusion protein of claim 3, wherein the protein further comprises an E2 polypeptide, a p7 polypeptide, an NS2 polypeptide, and optionally a core polypeptide.

6. The fusion protein of claim 3, wherein the protein further comprises an E1 polypeptide, an E2 polypeptide, a p7 polypeptide, an NS2 polypeptide, and optionally a core polypeptide.

7. The fusion protein of claim 3, wherein the protein further comprises an E2 polypeptide, and optionally a core polypeptide.

8. The fusion protein of claim 3, wherein the protein further comprises an E1 polypeptide, an E2 polypeptide, and optionally a core polypeptide.

9. The fusion protein of claim 1, wherein the protein comprises an E2 polypeptide, a modified NS3 polypeptide, and optionally a core polypeptide.

10. The fusion protein of claim 1, wherein the protein comprises an E1 polypeptide, an E2 polypeptide, a modified NS3 polypeptide, and optionally a core polypeptide.

11. The fusion protein of claim 1, wherein the polypeptides of (a) and (b) are derived from the same HCV isolate.

12. The fusion protein of claim 1, wherein at least one of the polypeptides present in the fusion is derived from a different isolate that the modified NS3 polypeptide.

13. An immunogenic fusion protein consisting essentially of, in amino terminal to carboxy terminal direction:
(a) a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, and an NS5a polypeptide;
(b) a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide and an NS5b polypeptide;
(c) an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, and an NS5a polypeptide;
(d) an E1 polypeptide, an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, and an NS5a polypeptide;
(e) an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide and an NS5b polypeptide;
(f) an E1 polypeptide, an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide and an NS5b polypeptide;
(g) an E2 polypeptide and a modified NS3 polypeptide comprising substitution of an amino acid corresponding to His-1083, Asp-1105 and/or Ser-1165, numbered relative to the full-length HCV-1 polyprotein such that protease activity is inhibited;
(h) an E1 polypeptide, an E2 polypeptide and a modified NS3 polypeptide comprising a substitution of an amino acid corresponding to His-1083, Asp-1105 and/or Ser-1165, numbered relative to the full-length HCV-1 polyprotein such that protease activity is inhibited;
(i) an E2 polypeptide, a p7 polypeptide, an NS2 polypeptide and a modified NS3 polypeptide comprising a substitution of an amino acid corresponding to His-1083, Asp-1105 and/or Ser-1165, numbered relative to the full-length HCV-1 polyprotein such that protease activity is inhibited; or
(j) an E1 polypeptide, an E2 polypeptide, a p7 polypeptide, an NS2 polypeptide and a modified NS3 polypeptide comprising a substitution of an amino acid corresponding to His-1083, Asp-1105 and/or Ser-1165, numbered relative to the full-length HCV-1 polyprotein such that protease activity is inhibited.

14. An immunogenic fusion protein consisting essentially of, in amino terminal to carboxy terminal direction:
(a) a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide, and a core polypeptide;
(b) a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide, an NS5b polypeptide and a core polypeptide;
(c) an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide and a core polypeptide;
(d) an E1 polypeptide, an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide and a core polypeptide;
(e) an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide, an NS5b polypeptide and a core polypeptide;
(f) an E1 polypeptide, an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, an NS4 polypeptide, an NS5a polypeptide, an NS5b polypeptide and a core polypeptide;
(g) an E2 polypeptide, a modified NS3 polypeptide comprising substitution of an amino acid corresponding to His-1083, Asp-1105 and/or Ser-1165, numbered relative to the full-length HCV-1 polyprotein such that protease activity is inhibited, and a core polypeptide;
(h) an E1 polypeptide, an E2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, and a core polypeptide;
(i) an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, and a core polypeptide; or
(j) an E1 polypeptide, an E2 polypeptide, a p7 polypeptide, an NS2 polypeptide, a modified NS3 polypeptide comprising a substitution of an amino acid corresponding to His-1083, Asp-1105 and/or Ser-1165, numbered relative to the full-length HCV-1 polyprotein such that protease activity is inhibited, and a core polypeptide.

15. A modified NS3 polypeptide comprising a substitution of an amino acid corresponding to His-1083, Asp-1105 and/or Ser-1165, numbered relative to the full-length HCV-1 polyprotein such that protease activity is inhibited when the modified NS3 polypeptide is present in an HCV fusion protein.

16. A composition comprising an immunogenic fusion protein according to claim 1 in combination with a pharmaceutically acceptable excipient.

17. A composition comprising an immunogenic fusion protein according to claim 13 in combination with a pharmaceutically acceptable excipient.

18. A composition comprising an immunogenic fusion protein according to claim 14 in combination with a pharmaceutically acceptable excipient.

19. A method of stimulating a cellular immune response in a vertebrate subject comprising administering a therapeutically effective amount of the composition of claim 16.

20. A method of stimulating a cellular immune response in a vertebrate subject comprising administering a therapeutically effective amount of the composition of claim 17.

21. A method of stimulating a cellular immune response in a vertebrate subject comprising administering a therapeutically effective amount of the composition of claim 18.

22. A method for producing a composition comprising combining the immunogenic fusion protein of claim 1 with a pharmaceutically acceptable excipient.

23. A method for producing a composition comprising combining the immunogenic fusion protein of claim 13 with a pharmaceutically acceptable excipient.

24. A method for producing a composition comprising combining the immunogenic fusion protein of claim 14 with a pharmaceutically acceptable excipient.

25. A polynucleotide comprising a coding sequence encoding a fusion protein according to claim 1.

26. A polynucleotide comprising a coding sequence encoding a fusion protein according to claim 13.

27. A polynucleotide comprising a coding sequence encoding a fusion protein according to claim 14.

28. A polynucleotide comprising a coding sequence encoding a polypeptide according to claim 15.

29. A recombinant vector comprising:
(a) the polynucleotide of claim 25; and
(b) at least one control element operably linked to said polynucleotide, whereby said coding sequence can be transcribed and translated in a host cell.

30. A recombinant vector comprising:
(a) the polynucleotide of claim 26; and
(b) at least one control element operably linked to said polynucleotide, whereby said coding sequence can be transcribed and translated in a host cell.

31. A recombinant vector comprising:
(a) the polynucleotide of claim 27; and
(b) at least one control element operably linked to said polynucleotide, whereby said coding sequence can be transcribed and translated in a host cell.

32. A recombinant vector comprising:
(a) the polynucleotide of claim 28; and
(b) at least one control element operably linked to said polynucleotide, whereby said coding sequence can be transcribed and translated in a host cell.

33. A host cell comprising the recombinant vector of claim 29.

34. A host cell comprising the recombinant vector of claim 30.

35. A host cell comprising the recombinant vector of claim 31.

36. A host cell comprising the recombinant vector of claim 32.

37. A method for producing an immunogenic fusion protein, said method comprising culturing a population of host cells according to claim 33 under conditions for producing said protein.

38. A method for producing an immunogenic fusion protein, said method comprising culturing a population of host cells according to claim 34 under conditions for producing said protein.

39. A method for producing an immunogenic fusion protein, said method comprising culturing a population of host cells according to claim 35 under conditions for producing said protein.

40. A method for producing a polypeptide, said method comprising culturing a population of host cells according to claim 36 under conditions for producing said polypeptide.
